SEQUENCE LISTING (12 sequences)

SEQ ID NO: 1 - DNA, 29 bp, Artificial
  Primer
atgcggatcccagtgtccctagctccgag

SEQ ID NO: 2 - DNA, 26 bp, Artificial
  primer
tttgtatttctttttgactacccagc

SEQ ID NO: 3 - DNA, 114 bp, Artificial
  Primer
aagaatactggaaagacggcaattgctgggtagtcaaaaagaaatacaagcagcagcagcagcagcagcagcagcagcagcagcagcagcagcaatgaaaaaaatgagtttgtt

SEQ ID NO: 4 - DNA, 36 bp, Artificial
  Primer
atgcaagcttttaagaaagtgcttccttatttattc

SEQ ID NO: 5 - DNA, 2480 bp, Artificial
  Fusion
ggatcccagtgtccctagctccgagaaaaaatccagagacaatttgtttctcatcaaggaagggtctttatactccgcatttaagtgaatctctcgcgcgccgcggaatgttttcggctgataaaaggaaatatggtatgacttctttttgaagtctctgatatgtgatccccgataagcgatatcaatatccagccttttttgatttaccttcatcacagctggcaccggatcatcgtcccatatatccttttttaattcacgcaagtcttttggatgaacaaacagctgataaagcggtaaattggattgattcttcatccataatcctccttacaaattttaggcttttatttttataagatctcagcggaacacttatacactttttaaaaccgcgcgtactatgagggtagtaaggatcttcatccttaacatatttttaaaaggaggatttcaaattgggccactattcccattctgacatcgaagaagcggtgaaatccgcaaaaaaagaaggtttaaaggattatttataccaagagcctcatggaaaaaaacgcagtcataaaaagtcgcaccgcactcacaaaaaatctcgcagccataaaaaatcatactgctctcacaaaaaatctcgcagtcacaaaaaatcattctgttctcacaaaaaatctcgcagccacaaaaaatcatactgctctcacaagaaatctcgcagccacaaaaaatcgtaccgttctcacaaaaaatctcgcagctataaaaaatcttaccgttcttacaaaaaatctcgtagctataaaaaatcttgccgttcttacaaaaaatctcgcagctacaaaaagtcttactgttctcacaagaaaaaatctcgcagctataagaagtcatgccgcacacacaaaaaatcttatcgttcccataagaaatactacaaaaaaccgcaccaccactgcgacgactacaaaagacacgatgattatgacagcaaaaaagaatactggaaagacggcaattgctgggtagtcaaaaagaaatacaaagcagcagcagcagcagcagcagcagcagcagcagcagcagcaatgaaaaaaatgagtttgtttcaaaatatgaaatcaaaacttctgccaatcgccgctgtttctgtccttacagctggaatctttgccggagctgagcttcagcaaacagaaaaggccagcgccaaaaaacaagacaaagctgagatcagaaatgtcattgtgatgataggcgacggcatggggacgccttacataagagcctaccgttccatgaaaaataacggtgacacaccgaataacccgaagttaacagaatttgaccggaacctgacaggcatgatgatgacgcatccggatgaccctgactataatattacagattcagcagcagccggaacagcattagcgacaggcgttaagacatataacaatgcaattggcgtcgataaaaacggaaaaaaagtgaaatctgtacttgaagaggccaaacagcaaggcaagtcaacagggcttgtcgccacgtctgaaattaaccacgccactccagccgcatatggcgcccacaatgaatcacggaaaaacatggaccaaatcgccaacagctatatggatgacaagataaaaggcaaacataaaatagacgtgctgctcggcggcggaaaatcttattttaaccgcaagaacagaaacttgacaaaggaattcaaacaagccggctacagctatgtgacaactaaacaagcattgaaaaaaaataaagatcagcaggtgctcgggcttttcgcagatggagggcttgctaaagcgctcgaccgtgacagtaaaacaccgtctctcaaagacatgacggtttcagcaattgatcgcctgaaccaaaataaaaaaggatttttcttgatggtcgaagggagccagattgactgggcggcccatgacaatgatacagtaggagccatgagcgaggttaaagattttgagcaggcctataaagccgcgattgaatttgcgaaaaaagacaaacatacacttgtgattgcaactgctgaccatacaaccggcggctttaccattggcgcaaacggggaaaagaattggcacgcagaaccgattctctccgctaagaaaacacctgaattcatggccaaaaaaatcagtgaaggcaagccggttaaagatgtgctcgcccgctatgccaatctgaaagtcacatctgaagaaatcaaaagcgttgaagcagctgcacaggctgacaaaagcaaaggggcctccaaagccatcatcaagatttttaatacccgctccaacagcggatggacgagtaccgatcataccggcgaagaagtaccggtatacgcgtacggccccggaaaagaaaaattccgcggattgattaacaatacggaccaggcaaacatcatatttaagattttaaaaactggaaaataaaagctt

SEQ ID NO: 6 - DNA, 2165 bp, Artificial
  Fusion
ggatcccagtgtccctagctccgagaaaaaatccagagacaatttgtttctcatcaaggaagggtctttatactccgcatttaagtgaatctctcgcgcgccgcggaatgttttcggctgataaaaggaaatatggtatgacttctttttgaagtctctgatatgtgatccccgataagcgatatcaatatccagccttttttgatttaccttcatcacagctggcaccggatcatcgtcccatatatccttttttaattcacgcaagtcttttggatgaacaaacagctgataaagcggtaaattggattgattcttcatccataatcctccttacaaattttaggcttttatttttataagatctcagcggaacacttatacactttttaaaaccgcgcgtactatgagggtagtaaggatcttcatccttaacatatttttaaaaggaggatttcaaattgggccactattcccattctgacatcgaagaagcggtgaaatccgcaaaaaaagaaggtttaaaggattatttataccaagagcctcatggaaaaaaacgcagtcataaaaagtcgcaccgcactcacaaaaaatctcgcagccataaaaaatcatactgctctcacaaaaaatctcgcagtcacaaaaaatcattctgttctcacaaaaaatctcgcagccacaaaaaatcatactgctctcacaagaaatctcgcagccacaaaaaatcgtaccgttctcacaaaaaatctcgcagctataaaaaatcttaccgttcttacaaaaaatctcgtagctataaaaaatcttgccgttcttacaaaaaatctcgcagctacaaaaagtcttactgttctcacaagaaaaaatctcgcagctataagaagtcatgccgcacacacaaaaaatcttatcgttcccataagaaatactacaaaaaaccgcaccaccactgcgacgactacaaaagacacgatgattatgacagcaaaaaagaatactggaaagacggcaattgctgggtagtcaaaaagaaatacaaagcagcagcagcagcagcagcagcagcagcagcagcagcagcagcagtgaatgaggaacatcatttcaaagtgactgcacacacggagacagatccggtcgcatctggcgatgatgcagcagatgacccggccatttgggttcatgaaaaacacccggaaaaaagcaagttgattacaacaaataagaagtcagggctcgttgtgtatgatttagacggaaaacagcttcattcttatgagtttggcaagctcaataatgtcgatctgcgctatgattttccattgaacggcgaaaaaattgatattgctgccgcatccaaccggtccgaaggaaaaaatacaattgaagtatatgcaatagacggggataaaggaaaattgaaaagcattacagatccgaaccatcctatttccaccaatatttctgaggtttatggattcagcttgtatcacagccagaaaacaggagcattttacgcattagtgacaggcaaacaaggggaatttgagcagtatgaaattgttgatggtggaaagggttatgtaacagggaaaaaggtgcgtgaatttaagttgaattctcagaccgaaggccttgttgcggatgatgagtacggaaacctatacatagcagaggaagatgaggccatctggaaatttaacgctgagcccggcggagggtcaaaggggcaggttgttgaccgtgcgacaggagatcatttgacagctgatattgaaggactgacaatctattatgcaccaaatggcaaaggatatctcatggcttcaagtcaaggaaataacagctatgcaatgtatgaacggcaggggaaaaatcgctatgtagccaactttgagattacagatggcgagaagatagacggtactagtgacacggatggtattgatgttctcggtttcggacttggcccaaaatatccgtacgggatttttgtggcgcaggacggcgaaaatattgataacggacaagccgtcaatcaaaatttcaaaattgtatcgtgggaacaaattgcacagcatctcggcgaaatgcctgatcttcataaacaggtaaatccgaggaagctgaaagaccgttctgacggctagtaaaagctt

SEQ ID NO: 7 - DNA, 2533 bp, Artificial
  Fusion
ggatcccacaggtgatgaaatgccgggtgggggacgcatggaggaccatatttccacctttgattatatgcctgaagatgaagtgataggtcatgatgtattagtaaaagtggagtggaggacaggccagaaaaaacagacagaagcaatcaaattacataagaagccatggtataaaaaatagtttatttgatgtatttgtgatcacattggtggtcacttttttatttgcggattcctaggcacagcaatctaagattctgcataggctgaaataaaatcttgttcatttctaaaacgaggtgcatgctgttggaacaacaaccaatcaatcatgaagacagaaacgtgccgcagcctattcgaagtgatggagctggagctattgatacaggcccgcgaaatataatacgggatattcaaaatccgaatatatttgttccgcctgttacagatgagggtatgattcctaacttgagattttcattctcagacgctcccatgaaattagatcacggcggctggtcaagagaaatcaccgtaagacagcttccgatttcgactgcgattgcaggtgtaaacatgagcttaactgcgggaggcgtccgcgagcttcattggcataagcaagcggagtgggcttatatgcttttgggacgggcacgtatcaccgctgttgaccaagacggacgaaatttcattgctgatgttggtcccggcgacctttggtacttcccggcaggaattccgcattccatacagggattggaacactgcgagtttctgctcgttttcgatgatgggaacttttctgagttttcaacgttaaccatttcagattggcttgcacacacaccaaaagatgttctgtctgcaaatttcggtgtcccggagaatgctttcaactctcttccgtctgagcaagtctatatctaccaagggaatgtgccgggatcagtcgccagtgaagacattcagtcaccatatggaaaagtccccatgacctttaaacacgagctgttaaatcaacccccaattcaaatgccaggggggagtgtacgttcagattgagcctggcgcgatgagagagcttcattggcatcccaatagcgatgagtggcaatattatctaacaggacagggacgaatgacggtatttatcggaaatgggactgcccgcacatttgattatagagcaggcgacgttggatacgtgccttctaatgccggacactatatacaaaacactggtacagaaacattatggtttttagaaatgttcaaaagtaaccgctatgcagatgtgtcactcaatcagtggatggcattgacgcctaaagaattagtacaaagcaacttgaatgctggatcagtcatgcttgattctctgcgcaagaagaaagtgcctgttgtgaaatatcccggtacggcagcagcagcagctagcgcagcagcagcagtgaatgaggaacatcatttcaaagtgactgcacacacggagacagatccggtcgcatctggcgatgatgcagcagatgacccggccatttgggttcatgaaaaacacccggaaaaaagcaagttgattacaacaaataagaagtcagggctcgttgtgtatgatttagacggaaaacagcttcattcttatgagtttggcaagctcaataatgtcgatctgcgctatgattttccattgaacggcgaaaaaattgatattgctgccgcatccaaccggtccgaaggaaaaaatacaattgaagtatatgcaatagacggggataaaggaaaattgaaaagcattacagatccgaaccatcctatttccaccaatatttctgaggtttatggattcagcttgtatcacagccagaaaacaggagcattttacgcattagtgacaggcaaacaaggggaatttgagcagtatgaaattgttgatggtggaaagggttatgtaacagggaaaaaggtgcgtgaatttaagttgaattctcagaccgaaggccttgttgcggatgatgagtacggaaacctatacatagcagaggaagatgaggccatctggaaatttaacgctgagcccggcggagggtcaaaggggcaggttgttgaccgtgcgacaggagatcatttgacagctgatattgaaggactgacaatctattatgcaccaaatggcaaaggatatctcatggcttcaagtcaaggaaataacagctatgcaatgtatgaacggcaggggaaaaatcgctatgtagccaactttgagattacagatggcgagaagatagacggtactagtgacacggatggtattgatgttctcggtttcggacttggcccaaaatatccgtacgggatttttgtggcgcaggacggcgaaaatattgataacggacaagccgtcaatcaaaatttcaaaattgtatcgtgggaacaaattgcacagcatctcggcgaaatgcctgatcttcataaacaggtaaatccgaggaagctgaaagaccgttctgacggctagtaaaagctt

SEQ ID NO: 8 - DNA, 3333 bp, Artificial
  Fusion
ggatcccacaggtgatgaaatgccgggtgggggacgcatggaggaccatatttccacctttgattatatgcctgaagatgaagtgataggtcatgatgtattagtaaaagtggagtggaggacaggccagaaaaaacagacagaagcaatcaaattacataagaagccatggtataaaaaatagtttatttgatgtatttgtgatcacattggtggtcacttttttatttgcggattcctaggcacagcaatctaagattctgcataggctgaaataaaatcttgttcatttctaaaacgaggtgcatgctgttggaacaacaaccaatcaatcatgaagacagaaacgtgccgcagcctattcgaagtgatggagctggagctattgatacaggcccgcgaaatataatacgggatattcaaaatccgaatatatttgttccgcctgttacagatgagggtatgattcctaacttgagattttcattctcagacgctcccatgaaattagatcacggcggctggtcaagagaaatcaccgtaagacagcttccgatttcgactgcgattgcaggtgtaaacatgagcttaactgcgggaggcgtccgcgagcttcattggcataagcaagcggagtgggcttatatgcttttgggacgggcacgtatcaccgctgttgaccaagacggacgaaatttcattgctgatgttggtcccggcgacctttggtacttcccggcaggaattccgcattccatacagggattggaacactgcgagtttctgctcgttttcgatgatgggaacttttctgagttttcaacgttaaccatttcagattggcttgcacacacaccaaaagatgttctgtctgcaaatttcggtgtcccggagaatgctttcaactctcttccgtctgagcaagtctatatctaccaagggaatgtgccgggatcagtcgccagtgaagacattcagtcaccatatggaaaagtccccatgacctttaaacacgagctgttaaatcaacccccaattcaaatgccaggggggagtgtacgaattgtggattcttctaacttcccaatttcaaaaacgatagccgctgcacttgttcagattgagcctggcgcgatgagagagcttcattggcatcccaatagcgatgagtggcaatattatctaacaggacagggacgaatgacggtatttatcggaaatgggactgcccgcacatttgattatagagcaggcgacgttggatacgtgccttctaatgccggacactatatacaaaacactggtacagaaacattatggtttttagaaatgttcaaaagtaaccgctatgcagatgtgtcactcaatcagtggatggcattgacgcctaaagaattagtacaaagcaacttgaatgctggatcagtcatgcttgattctctgcgcaagaagaaagtgcctgttgtgaaatatcccggtacggcagcagcagctagcgcagcagcagcagcaatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccgggatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgataaaagctt

SEQ ID NO: 9 - DNA, 2543 bp, Artificial
  Fusion
ggatccttattttgtttgtgggttttttagtatttgggcctgataaactgccggcgcttggccgtgcagcaggaaaagccttatcagaatttaaacaagcaacaagcggactgactcaggatatcagaaaaaatgactcagaaaacaaagaagacaaacaaatgtaggataaatcgtttgggccgatgaaaaatcggctctttattttgatttgtttttgtgtcatctgtctttttctatcatttggacagcccttttttccttctatgattttaactgtccaagccgcaaaatctactcgccgtataataaagcgtagtaaaaataaaggaggagtatatatgggttattacaaaaaatacaaagaagagtattatacggtcaaaaaaacgtattataagaagtattacgaatatgataaaaaagattatgactgtgattacgacaaaaaatatgatgactatgataaaaaatattatgatcacgataaaaaagactatgattatgttgtagagtataaaaagcataaaaaacactacgcagcagcagcagcagcagcagcagcagcaatgtccttccttggtggcgccgagtgctcgacggcgggcaatccgttgactcagttcaccaagcacgtccaagatgataagtccctacagagagatcgcctcgtggggcgaggcccaggaggcatgcaagaaggcatgcggtcccggggtatgatgggaggacaagatcagatgatggacgaattcgcccaacaacccggccagatccccggtgctcccccgcaaccgttcgctatggaacagctgcgacgcgagctagatcagttccaaaccacacctccgaggacgggctcccccggctgggcggccgagttcgatgcgggcgagcatgcccggatggaggctgcgtttgccgggccccagggccccatgatgaataatgcgtcgggatttacgcccgcggagtttgcccggttccagcagcagagtcgggctggcatgcctcagacggctaaccatgtggcgtctgccccgtcgccgatgatggctgggtaccagcggcccatgggtatgggagggtatatgggcatgggtggaatggggatgatgccgcagacatttaacccgatggcgatgcagcagcagccggcagaggcgactacgcaggacaagggcaagggacgcatggtggagctggacgacgagaactgggaggcacagtttgccgagatggagacggcggatacccagaaattggacgatgaggccaacgcagctgtggaggcagagctgaatgatctggataggtcagtcccccaagattcgggcgatagtgcctttgaaagcgtgtggcaacgggtccaagctgagaccgcaacaaacaggaaactggccgagggcgagaccgactttaacattgacgacaatctgcatatgggtgagatgggcgaatgggacggattcgatacgcttaacacgcgcttccggaaccctcaactaggcgattatatgttcgaagaagataacgtgttccggagcgtgagcaatcctttcgaagagggagtgaagatcatgcgcgagggtggaaacctctccctggctgccttggctttcgaggcggcagtccagaaagatcctcaacatgtccaggcctggaccatgctgggatcggctcaggcgcagaacgagaaggagcttcccgccatcagagcgctggagcaggcacttaagattgatgctaacaatctggatgcgctgatgggactggctgtttcctacaccaacgagggctatgactcgacatcgtaccgcactttggagcgttggctgtcagtcaagtacccccagattatcaaccctaatgatgtttcatcggaagccgacttgggctttacggaccgccagctcctgcacgaccgtgtcaccgatctcttcatccaggctgctcagctgtcgccatctggcgagcaaatggacccggacgtccaggtcggtcttggcgttctcttctactgcgcagaggagtatgacaaggcggtcgattgcttctctgctgcgttggcgtccacggaatccggaacgtcgaaccaacaggagcagctccacctgctgtggaaccgtctgggtgctacgcttgccaactcgggtcgctccgaggaggcgatcgaggcctacgagcaggcgctgaacatcaatcccaacttcgtccgggcacggtacaacctgggtgtgtcgtgcatcaacatcggctgctacccagaagccgcgcaacacctgctgggagcgctatcgatgcaccgggtggttgagcaggaaggtcgagagcgggcacgtgagattgttgggggcgagggtggcattgacgacgagcagctggatcgcatgattcatgtcagccagaatcagagtaccaacctgtacgacacgttgcggcgagtatttagccagatgggacgacgcgatctggctgatcaggtggtggcggggatggatgtcaatgtgttccgacgggagtttgagttctaataaaagctt

SEQ ID NO: 10 - DNA, 3433 bp, Artificial
  Fusion
ggatcccacaggtgatgaaatgccgggtgggggacgcatggaggaccatatttccacctttgattatatgcctgaagatgaagtgataggtcatgatgtattagtaaaagtggagtggaggacaggccagaaaaaacagacagaagcaatcaaattacataagaagccatggtataaaaaatagtttatttgatgtatttgtgatcacattggtggtcacttttttatttgcggattcctaggcacagcaatctaagattctgcataggctgaaataaaatcttgttcatttctaaaacgaggtgcatgctgttggaacaacaaccaatcaatcatgaagacagaaacgtgccgcagcctattcgaagtgatggagctggagctattgatacaggcccgcgaaatataatacgggatattcaaaatccgaatatatttgttccgcctgttacagatgagggtatgattcctaacttgagattttcattctcagacgctcccatgaaattagatcacggcggctggtcaagagaaatcaccgtaagacagcttccgatttcgactgcgattgcaggtgtaaacatgagcttaactgcgggaggcgtccgcgagcttcattggcataagcaagcggagtgggcttatatgcttttgggacgggcacgtatcaccgctgttgaccaagacggacgaaatttcattgctgatgttggtcccggcgacctttggtacttcccggcaggaattccgcattccatacagggattggaacactgcgagtttctgctcgttttcgatgatgggaacttttctgagttttcaacgttaaccatttcagattggcttgcacacacaccaaaagatgttctgtctgcaaatttcggtgtcccggagaatgctttcaactctcttccgtctgagcaagtctatatctaccaagggaatgtgccgggatcagtcgccagtgaagacattcagtcaccatatggaaaagtccccatgacctttaaacacgagctgttaaatcaacccccaattcaaatgccaggggggagtgtacgttcagattgagcctggcgcgatgagagagcttcattggcatcccaatagcgatgagtggcaatattatctaacaggacagggacgaatgacggtatttatcggaaatgggactgcccgcacatttgattatagagcaggcgacgttggatacgtgccttctaatgccggacactatatacaaaacactggtacagaaacattatggtttttagaaatgttcaaaagtaaccgctatgcagatgtgtcactcaatcagtggatggcattgacgcctaaagaattagtacaaagcaacttgaatgctggatcagtcatgcttgattctctgcgcaagaagaaagtgcctgttgtgaaatatcccggtacggcagcagcagcagctagcgcagcagcagcaatgtccttccttggtggcgccgagtgctcgacggcgggcaatccgttgactcagttcaccaagcacgtccaagatgataagtccctacagagagatcgcctcgtggggcgaggcccaggaggcatgcaagaaggcatgcggtcccggggtatgatgggaggacaagatcagatgatggacgaattcgcccaacaacccggccagatccccggtgctcccccgcaaccgttcgctatggaacagctgcgacgcgagctagatcagttccaaaccacacctccgaggacgggctcccccggctgggcggccgagttcgatgcgggcgagcatgcccggatggaggctgcgtttgccgggccccagggccccatgatgaataatgcgtcgggatttacgcccgcggagtttgcccggttccagcagcagagtcgggctggcatgcctcagacggctaaccatgtggcgtctgccccgtcgccgatgatggctgggtaccagcggcccatgggtatgggagggtatatgggcatgggtggaatggggatgatgccgcagacatttaacccgatggcgatgcagcagcagccggcagaggcgactacgcaggacaagggcaagggacgcatggtggagctggacgacgagaactgggaggcacagtttgccgagatggagacggcggatacccagaaattggacgatgaggccaacgcagctgtggaggcagagctgaatgatctggataggtcagtcccccaagattcgggcgatagtgcctttgaaagcgtgtggcaacgggtccaagctgagaccgcaacaaacaggaaactggccgagggcgagaccgactttaacattgacgacaatctgcatatgggtgagatgggcgaatgggacggattcgatacgcttaacacgcgcttccggaaccctcaactaggcgattatatgttcgaagaagataacgtgttccggagcgtgagcaatcctttcgaagagggagtgaagatcatgcgcgagggtggaaacctctccctggctgccttggctttcgaggcggcagtccagaaagatcctcaacatgtccaggcctggaccatgctgggatcggctcaggcgcagaacgagaaggagcttcccgccatcagagcgctggagcaggcacttaagattgatgctaacaatctggatgcgctgatgggactggctgtttcctacaccaacgagggctatgactcgacatcgtaccgcactttggagcgttggctgtcagtcaagtacccccagattatcaaccctaatgatgtttcatcggaagccgacttgggctttacggaccgccagctcctgcacgaccgtgtcaccgatctcttcatccaggctgctcagctgtcgccatctggcgagcaaatggacccggacgtccaggtcggtcttggcgttctcttctactgcgcagaggagtatgacaaggcggtcgattgcttctctgctgcgttggcgtccacggaatccggaacgtcgaaccaacaggagcagctccacctgctgtggaaccgtctgggtgctacgcttgccaactcgggtcgctccgaggaggcgatcgaggcctacgagcaggcgctgaacatcaatcccaacttcgtccgggcacggtacaacctgggtgtgtcgtgcatcaacatcggctgctacccagaagccgcgcaacacctgctgggagcgctatcgatgcaccgggtggttgagcaggaaggtcgagagcgggcacgtgagattgttgggggcgagggtggcattgacgacgagcagctggatcgcatgattcatgtcagccagaatcagagtaccaacctgtacgacacgttgcggcgagtatttagccagatgggacgacgcgatctggctgatcaggtggtggcggggatggatgtcaatgtgttccgacgggagtttgagttctaataaaagctt

SEQ ID NO: 11 - DNA, 1968 bp, Aspergillus niger
atgtctttccttggcggtgctgagtgctcaactgccggaaacccgctgactcaattcacaaagcacgttcaggatgacaaatcacttcagcgtgaccgtcttgtcggacgcggaccgggcggtatgcaggaaggcatgcgttctcgcggtatgatgggcggacaggatcaaatgatggatgaattcgcacagcagccaggtcaaatcccaggtgcgccgcctcagccatttgcgatggagcagcttcgccgtgagcttgatcaattccaaacaactccacctcgtactggttctccaggctgggcagctgaattcgacgctggtgagcacgcccgtatggaagctgctttcgccggaccgcaaggtccaatgatgaacaacgcttcaggcttcactccagctgaattcgcccgtttccagcagcagtctcgtgcgggtatgcctcaaacggcaaaccacgttgcaagtgctccttctccaatgatggctggttatcagcgtccgatgggtatgggcggatacatgggtatgggcggtatgggtatgatgcctcaaacgttcaacccaatggcgatgcagcagcagcctgctgaagcaacaactcaagacaaaggtaaaggccgtatggttgagcttgatgacgaaaactgggaagctcaattcgctgaaatggaaactgctgacactcaaaagctagatgatgaagcaaacgctgctgttgaagctgagctgaacgatcttgaccgttctgttcctcaggattcaggtgacagtgcgtttgaatctgtttggcagcgtgttcaggctgaaactgcaacaaaccgcaagctggctgaaggtgaaactgacttcaacatcgatgacaaccttcacatgggtgaaatgggtgagtgggacggtttcgacactttaaacactcgtttccgcaaccctcagcttggtgattacatgttcgaagaagacaacgtattccgttctgtatcaaacccatttgaagaaggcgtaaaaatcatgcgtgaaggcggaaacctttctcttgctgcgcttgcgtttgaagctgctgttcaaaaagaccctcagcacgttcaggcttggacgatgcttggttctgctcaagctcaaaacgaaaaagagcttcctgccatccgtgcgcttgagcaggctttaaaaatcgatgctaacaaccttgatgctttaatgggtcttgctgtcagctacacaaatgaaggctatgacagcacttcttaccgtacgcttgagcgctggctttctgtaaaataccctcaaatcatcaacccaaacgatgtatcaagtgaagctgatcttggcttcactgaccgtcaattgcttcatgaccgtgtaactgatttgttcattcaagctgcacagctttctccatctggtgagcaaatggaccctgatgttcaagtaggtcttggtgtactattctactgtgctgaagaatacgataaagcggttgactgcttctctgctgctcttgcttcaactgaaagcggaacttcaaaccagcaagagcagcttcatttgctatggaaccgtcttggtgcgacgcttgcaaacagcggacgcagtgaagaagcgatcgaagcatatgagcaggcgctgaacatcaacccaaacttcgttcgtgcgcgttacaacctaggtgtatcttgtatcaacatcggctgttatcctgaagcggcacagcatttgcttggtgctttatcaatgcaccgtgttgttgagcaggaaggccgtgagcgtgcgcgtgaaatcgtcggcggtgaaggcggtatcgatgatgagcagcttgaccgcatgattcacgtttctcaaaaccaatctacaaacctatatgatacgcttcgccgtgtattctctcaaatgggcagaagagatcttgctgatcaggttgtagcgggtatggatgtaaacgtattccgtcgtgagtttgaattctaataa

SEQ ID NO: 12 - protein, 654 aa, Aspergillus niger
MSFLGGAECSTAGNPLTQFTKHVQDDKSLQRDRLVGRGPGGMQEGMRSRGMMGGQDQMMDEFAQQPGQIPGAPPQPFAMEQLRRELDQFQTTPPRTGSPGWAAEFDAGEHARMEAAFAGPQGPMMNNASGFTPAEFARFQQQSRAGMPQTANHVASAPSPMMAGYQRPMGMGGYMGMGGMGMMPQTFNPMAMQQQPAEATTQDKGKGRMVELDDENWEAQFAEMETADTQKLDDEANAAVEAELNDLDRSVPQDSGDSAFESVWQRVQAETATNRKLAEGETDFNIDDNLHMGEMGEWDGFDTLNTRFRNPQLGDYMFEEDNVFRSVSNPFEEGVKIMREGGNLSLAALAFEAAVQKDPQHVQAWTMLGSAQAQNEKELPAIRALEQALKIDANNLDALMGLAVSYTNEGYDSTSYRTLERWLSVKYPQIINPNDVSSEADLGFTDRQLLHDRVTDLFIQAAQLSPSGEQMDPDVQVGLGVLFYCAEEYDKAVDCFSAALASTESGTSNQQEQLHLLWNRLGATLANSGRSEEAIEAYEQALNINPNFVRARYNLGVSCINIGCYPEAAQHLLGALSMHRVVEQEGRERAREIVGGEGGIDDEQLDRMIHVSQNQSTNLYDTLRRVFSQMGRRDLADQVVAGMDVNVFRREFEF

(19) United States
(12) Patent Application Publication
(10) Pub. No.: US 2014/0315278 A1
(43) Pub. Date: Oct. 23, 2014

(54) SPORE SURFACE DISPLAYS OF BIOACTIVE MOLECULES

(71) Applicant: DSM IP Assets B.V., Heerlen (NL)

(72) Inventors: Adriano O. HENRIQUES, Cascais (PT); Ghislain Schyns, Aesch (CH); Thibaut José Wenzel, Leiden (NL); Sebastien Potot, Hegenheim (FR)

(73) Assignee: DSM IP Assets B.V., Heerlen (NL)

(21) Appl. No.: 14/225,787

(22) Filed: Mar. 26, 2014

Related U.S. Application Data
(63) Continuation of application No. 12/375,976, filed on Nov. 5, 2009, now abandoned.

(30) Foreign Application Priority Data
Aug. 9, 2006 (EP) 06016599.0

(51) Int. Cl. C12N 15/75

(52) U.S. Cl. CPC C12N 15/75
USPC 435/252.31

(57) ABSTRACT

This invention discloses novel bacterial spore systems. Genetically modified or genetically engineered viable spore systems expressing bioactive polypeptides, for example bacteriocins and/or enzymatically active feed enzymes, at the spore surface, have a great potential use in animal feeding. Genetically modified or “genetically engineered inert spore systems expressing affinity ligands or immobilized enzymes at the surface have a great potential use in biocatalysis and in the construction of biocatalytic films. Especially the resistance to harsh chemicals, desiccation, strong pressure, or high temperatures allows the spores to be a potentially valuable tool for the display of bioactive molecules, like biocatalytic enzymes or bioactive feed enzymes that must survive harsh conditions to deliver their full potential. Finally, instead of translational fusions to spore structural genes, passenger bioactive polypeptides, as for example enzymes, bacteriocins, affinity ligands, can also be fused to spore-specific surface enzymes, for example to spore specific enzymes as mentioned herein above.

This application is a continuation of application Ser. No. 12/375,976 (pending), filed Nov. 5, 2009 (published as US 2010-0055244 A1), which is a U.S. national phase of International Application No. PCT/EP2007/007052, filed Aug. 9, 2007, which designated the U.S. and claims priority to European Patent Application No. 06016599.0, filed Aug. 9, 2006, the entire contents of each of which are hereby incorporated by reference.

The present invention relates to the display of bioactive molecules at the surface of spores for both in vitro and in vivo applications.

During the last ten years microbial surface display (part of the bio-nanotechnology field) has increasingly become a tool of choice to display peptides or proteins of biotechnological interest on natural nanostructures for a commercial purpose. Biological applications include the development of bio-adsorbents, the presentation of antigens for vaccines, or the preparation of combinatorial epitope libraries. Surface display requires only the synthesis of a hybrid protein that consists of a passenger protein of commercial interest fused to a carrier protein, which anchors it onto the biological surface (cell wall or membrane). A good carrier protein requires the following characteristics: i) a targeting signal that directs it to the biological surface; ii) a strong anchoring motif; iii) resistance to proteases; and iv) compatibility to foreign sequences to be fused. Originally, the carrier protein was chosen amongst surface or membrane proteins, e.g. OmpA for Gram-negative bacteria or the Protein A for Gram-positive bacteria. The disadvantages of these display systems are that these proteins were not very stable and tended to be inactivated under conditions that are regularly used in biotechnological and chemical processes.

Recently, another nanostructure has emerged as a novel surface of choice for display: the spore coat from Bacillus subtilis and other related genera. Bacilli and Clostridia have the ability to undergo a complex differentiation process under nutrient deprivation or hostile conditions. This process, called sporulation, ends with the formation of an extremely resistant structure named the spore. When conditions become conductive for growth, the spores germinate to re-generate vegetative cells which follow a classical growth and division cyclic pattern. Spore consists of a central compartment, the spore core, which contains a copy of the chromosome. The spore core is surrounded by a thin inner layer membrane of peptidoglycan that creates the germ cell, itself surrounded by a thicker layer of peptidoglycan, called the cortex. Outside of the cortex, a multilayered protein shell, the coat, provides unique resistance characteristics. B. subtilis coat is formed by the ordered assembly of over 40 polypeptides. Some of these have enzymatic activity, like oxdD, which encodes an oxalate decarboxylase, cotA which encodes a laccase, yvdO which encodes a phospholipase, cotQ which encodes a reticuline-oxidase or tgl which encodes a transglutaminase. In contrast to vegetative cells, the spore coat proteins allow spores to be very resistant to harsh chemicals, desiccation, strong pressure, or high temperatures.

An example of B. subtilis spore is disclosed in WO 2005/028556. Known spores which show synthetic enzymatic activity displayed at the spore surfaces are very limited and refer to the use as diagnostic system or pharmaceutical drug, e.g. vaccine delivery systems. Examples reported are displays of β-galactosidases, which were fused to part of CotC, to CotD, CotE, CotG or InhA (WO1996/23063; US2004/0171065; WO2005/028654), and displays of lipases, which were inserted in frame within CotC or fused to part of CotC (US2002/0150594) or displays of carboxymethylcellulases, which were fused to the exosporium protein InhA.

It has now been found surprisingly that under certain conditions spore systems, as described in general herein above, can be used in the food and feed industry, preferably in animal feeding. More precisely, applicant has found the following: genetically modified or genetically engineered viable spore systems expressing bioactive polypeptides, for example bacteriocins and/or enzymatically active feed enzymes, at the spore surface, have a great potential use in animal feeding. Further, it has been found that genetically modified or “genetically engineered” inert spore systems expressing affinity ligands or immobilized enzymes at the surface have a great potential use in biocatalysis and in downstream purification processes. Especially the resistance to harsh chemicals, desiccation, strong pressure, or high temperatures allows the spores to be a potentially valuable tool for the display of bioactive molecules, like biocatalytic enzymes or bioactive feed enzymes that must survive harsh reaction conditions to deliver their full potential. Finally, applicant has found that instead of translational fusions to spore structural genes as it is known from the prior art described above, passenger bioactive polypeptides, as for example enzymes, bacteriocins, affinity ligands, can also be fused to spore-specific enzymes, for example to surface enzymes as mentioned herein above.

The terms “spore” and “spore system” as used herein are equivalent expressions and denote differentiated resistant structures that come from differentiation of microbial vegetative cells under hostile physical or chemical conditions such as, but not limited to, extreme pH, heat, pressure, desiccation or an extract/mixture containing said structures, wherein the spore is derived from a parent spore-forming organisms.

The spore which can be used in the present invention may be publicly available from different sources, e.g., Deutsche Sammlung von Mikroorganismen and Zellkulturen (DSMZ), Mascheroder Weg 1B, D-38124 Braunschweig, Germany, American Type Culture Collection (ATCC), P.O. Box 1549, Manassas, Va. 20108 USA or Culture Collection Division, NITE Biological Resource Center, 2-5-8, Kazusakamatari, Kisarazu-shi, Chiba, 292-0818, Japan (formerly: Institute for Fermentation, Osaka (IFO), 17-85, Juso-honmachi 2-chome, Yodogawa-ku, Osaka 532-8686, Japan), or alternatively from well characterized (wild) isolates, which sporulate with higher efficiency that laboratory strains. Examples of preferred spores are spores of Bacilli, Sporolactobacilli and Clostridia, for example bacterial spores of B. subtilis.

It is a first object of the present invention to provide a new genetically modified, inert spore which is unable to germinate wherein said spore is genetically modified to expose at its surface affinity ligands and/or biocatalysts, for example immobilized enzymes.

The term “genetically modified” or “genetically engineered” means the scientific alteration of the structure of genetic material in a living organism. It involves the production and use of recombinant DNA. More in particular it is used to delineate the genetically engineered or modified organism from the naturally occurring organism by forming a genetic DNA construct, wherein the genetic DNA construct comprises a first DNA portion encoding the desired target protein (including but not limited to affinity ligand, bioactive polypeptide, or enzyme) and a second DNA portion encoding a carrier herein also called spore coat protein, which construct, when transcribed and translated, expresses a fusion protein between the carrier and the target protein or peptide. Genetic engineering may be done by a number of techniques known in the art, such as gene replacement, gene amplification, gene disruption, transfection, transformation using plasmids, viruses, or other vectors. A genetically modified organism, e.g. genetically modified microorganism, is also often referred to as a recombinant organism, e.g. recombinant microorganism.

The DNA encoding portion of the construct encoding the carrier may be selected from:
a) the group of spore structural genes comprising cotC (encoding spore inner coat protein CotC), cotD (encoding spore inner coat protein CotD), cotB (encoding spore outer coat protein CotB), cotE (encoding spore outer coat protein CotE), cotF (encoding spore coat protein CotF), cotG (encoding spore coat protein CotG), cotN (encoding spore protein CotN), cotS (encoding spore coat protein CotS), cotT (encoding spore inner coat protein CotT), cotV (encoding spore coat protein CotV), cotW (encoding spore coat protein CotW), cotX (encoding spore coat protein CotX), cotY (encoding spore coat protein CotY), cotZ (encoding spore coat protein CotZ), cotH (encoding spore inner coat protein CotH), cotJA (encoding spore coat protein CotJA), cot JC (encoding spore coat protein CotJC), cotK (encoding spore protein CotK), cotL (encoding spore protein CotL), cotM (encoding spore outer coat protein CotM), spoIVA (encoding spore assembly protein SpoIVA), spoVID (encoding spore assembly protein SpoVID), or any other gene coding for a protein whose assembly at the surface of the developing spore has been shown to be dependent on spoIVA, spoVID, safA or cotE.
b) the group of spore specific enzymes comprising cotA (encoding a laccase), oxdD (encoding an oxalate decarboxylase), cotQ (encoding a reticuline oxidase-like protein), tgl (encoding a transglutaminase), or the product of any other gene which resembles a known enzymes, and whose assembly at the surface of the developing spore has been shown to be dependent on spoIVA, spoVID, safA or cotE.

The DNA encoding portion of the construct encoding the target may be selected from but not limited to affinity ligands, bioactive polypeptides, biocatalysis enzymes or any other enzymes.

The term “biocatalysis” as used herein denotes a chemical reaction mediated by a biological molecule, called biocatalyst, and which is able to initiate or modify the rate of the reaction in vivo (within a living system) or in vitro (within a reconstituted system). Enzymes are examples of biocatalysts.

Soluble enzymes can be immobilized following different procedures mainly in order to reuse and to stabilize them. Examples of immobilized enzymes are Candida rugosa lipase (CRL) encapsulated without carrier, trypsin, Candida Antarctica lipase (CalB) or penicillin G acylase cross-linked to macromolecule (e.g. polyethylene glycol or dextran sulfate) or alkylsulfatase on anionic exchangers.

An example of an affinity ligand with in vivo biological relationship with the target protein is the A. niger PTS-1 affine Pex5 protein. Pex5 is the receptor of PTS-1 [McCollum et al., J. Cell Biol. 121, 761-774 (1993)]. PTS-1 is a C-terminal tri-peptide extension of a protein promoting peroxisomal localization of the protein. The C-terminal tri-peptide PTS-1 can be a variant of [PAS]-[HKR]-[L] as described in Emanuelsson et al., J. Mol. Biol. (2003) 330, 443-456. Preferably PTS-1 is -SKL or -PRL. The term “affinity ligand” as used here denotes not only molecules that have biological relationship in vivo with the target protein but also a variety of other ligand such as fusion proteins or affinity tags. Examples of affinity tags or fusion proteins are the maltose binding protein (MBP) that interacts with cross-linked amylose and is eluted with maltose, polyhistidine tags that consists of 6 His residues binding to chelated Ni²⁺ or FLAG tag that is a eight amino acid hydrophilic peptide that binds to a specific antibody linked onto a column.

Inert spore are spores which are unable to germinate and recreate vegetative life. Methods to generate Bacillus subtilis non-germinating strain are well known from people skilled in the art. Inert spores according to this aspect of the invention are for example used “in vitro” and allow for example an alternative option to expensive classical systems of immobilized enzymes. They primarily have the advantage of spore resistance to harsh chemical conditions.

In a further aspect the invention relates to the use of inert spore systems expressing at their surface affinity ligands and/or bicocatalysts in biocatalysis and for the production of bioactive materials comprising such spore systems. An example of use of an inert spore system expressing at the surface the affinity ligand A. niger Pex5 protein, is affinity purification of proteins comprising a C-terminal PTS-1 tag. The PTS-1 tagged proteins are preferably produced by the method described in WO2006/040340A2.

It is another object of the present invention to provide a genetically modified, viable spore which is able to germinate wherein said spore is genetically modified to produce an enzyme or a bioactive polypeptide upon germination into a vegetative cell.

Examples of enzymes which can be used in such a system are enzymes for the food industry and feed enzymes. Preferred feed enzymes are selected from amongst phytase (EC 3.1.3.8 or 3.1.3.26), xylanase (EC 3.2.1.8); galactanase (EC 3.2.1.89); alpha-galactosidase (EC 3.2.1.22); protease (EC 3.4.), phospholipase A1 (EC 3.1.1.32); phospholipase A2 (EC 3.1.1.4); lysophospholipase (EC 3.1.1.5); phospholipase C (EC 3.1.4.3); phospholipase D (EC 3.1.4.4); amylase such as, for example, alpha-amylase (EC 3.2.1.1); and/or beta-glucanase (EC 3.2.1.4 or EC 3.2.1.6).

Bioactive polypeptides which can be used for the fusion according to the invention are antimicrobial and antifungal polypeptides. Examples of antimicrobial peptides (AMP's) are CAP18, Leucocin A, Tritrpticin, Protegrin-1, Thanatin, Defensin, Lactoferrin, Lactoferricin, and Ovispirin such as Novispirin, Plectasins, and Statins, including the compounds and polypeptides disclosed in WO 03/044049 and WO 03/048148, as well as variants or fragments of the above that retain antimicrobial activity. Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

Display on viable/live spores allows amplification of spore population in situ through the sporulation-germination-vegetative growth cycle. Therefore, such a spore system according to the invention allows a continuously deliver of fresh enzymes. It is a further advantage of such systems that the spores are resistant to difficult conditions of digestive tracts and that they are easy to produce and can be made at low costs.

In a preferred embodiment of the invention, the genetic modification is accomplished by transformation of a precursor cell using a vector containing the chimeric gene, using standard methods known to persons skilled in the art and then inducing the precursor cell to produce spores according to the invention. Further, the system may be constructed as such, that the gene construct may be under the control of one or more inducible promoter. The gene construct may have one or more enhancer elements or upstream activator sequences and the like associated with it. The gene construct may also comprise an inducible expression system. The inducible expression system is such that when said spore germinates into a vegetative cell, the active polypeptide or enzyme is not expressed unless exposed to an external stimulus e.g. pH.

If the spore system according to the invention expresses a feed enzyme on the spore surface, the spore germinates in the intestinal tract. More preferably the spore germinates in the duodenum and/or the jejunum of the intestinal tract.

In a further aspect of the invention the viable spore can be constructed as such that it displays a combination of both feed enzyme and bioactive polypeptide.

It is a further object of the invention to provide a composition comprising spores which express bioactive peptides and/or enzymes on their surface.

In a preferred embodiment of the invention, the composition comprises spores of the invention which express a feed enzyme as for example phytase (EC 3.1.3.8 or 3.1.3.26).

Particular examples of compositions of the invention are the following:
an animal feed additive comprising (a) a spore expressing a feed enzyme according to the invention; and (b) at least one fat-soluble vitamin, (c) at least one water-soluble vitamin, (d) at least one trace mineral, and/or (e) at least one macro mineral; and
an animal feed composition having a crude protein content of 50 to 800 g/kg and comprising a spore expressing a feed enzyme according to the invention.

The so-called premixes are examples of animal feed additives of the invention. A premix designates a preferably uniform mixture of one or more micro-ingredients with diluent and/or carrier. Premixes are used to facilitate uniform dispersion of micro-ingredients in a larger mix.

The term animal includes all animals. Examples of animals are non-ruminants, and ruminants. Ruminant animals include, for example, animals such as sheep, goat, and cattle, e.g. cow such as beef cattle and dairy cows. In a particular embodiment, the animal is a non-ruminant animal. Non-ruminant animals include mono-gastric animals, e.g. pig or swine (including, but not limited to, piglets, growing pigs, and sows); poultry such as turkeys, ducks and chickens (including but not limited to broiler chicks, layers); fish (including but not limited to salmon, trout, tilapia, catfish and carp); and crustaceans (including but not limited to shrimp and prawn).

The term feed or feed composition means any compound, preparation, mixture, or composition suitable for, or intended for intake by an animal.

Further, optional, feed-additive ingredients are colouring agents, e.g. carotenoids such as beta-carotene, astaxanthin, and lutein; aroma compounds; stabilisers; antimicrobial peptides; polyunsaturated fatty acids and/or reactive oxygen generating species.

In a particular embodiment, the animal feed additive of the invention is intended for being included (or prescribed as having to be included) in animal diets or feed at levels of 0.01 to 10.0%; more particularly 0.05 to 5.0%; or 0.2 to 1.0% (% meaning g additive per 100 g feed). This is so in particular for premixes.

Animal feed compositions or diets have a relatively high content of protein. Poultry and pig diets can be characterised as indicated in Table B of WO 01/58275, columns 2-3. Fish diets can be characterised as indicated in column 4 of this Table B. Furthermore such fish diets usually have a crude fat content of 200-310 g/kg. WO 01/58275 corresponds to U.S. Ser. No. 09/779,334 which is hereby incorporated by reference.

An animal feed composition according to the invention has a crude protein content of 50-800 g/kg, and furthermore comprises at least one spore strain as described and/or claimed herein.

Furthermore, or as an alternative to the crude protein content indicated above, the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table B of WO 01/58275 (R. 2-5).

Crude protein is calculated as nitrogen (N) multiplied by a factor 6.25, i.e. Crude protein (g/kg)=N (g/kg)×6.25. The nitrogen content is determined by the Kjeldahl method (A.O.A.C., 1984, Official Methods of Analysis 14th ed., Association of Official Analytical Chemists, Washington D.C.).

Metabolisable energy can be calculated on the basis of the NRC publication Nutrient requirements in swine, ninth revised edition 1988, subcommittee on swine nutrition, committee on animal nutrition, board of agriculture, national research council. National Academy Press, Washington, D.C., pp. 2-6, and the European Table of Energy Values for Poultry Feed-stuffs, Spelderholt centre for poultry research and extension, 7361 DA Beekbergen, The Netherlands. Grafisch bedrijf Ponsen & looijen bv, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In a particular embodiment, the animal feed composition of the invention contains at least one vegetable protein or protein source. It may also contain animal protein, such as Meat and Bone Meal, and/or Fish Meal, typically in an amount of 0-25%. The term vegetable proteins as used herein refers to any compound, composition, preparation or mixture that includes at least one protein derived from or originating from a vegetable, including modified proteins and protein-derivatives. In particular embodiments, the protein content of the vegetable proteins is at least 10, 20, 30, 40, 50, or 60% (w/w).

Vegetable proteins may be derived from vegetable protein sources, such as legumes and cereals, for example materials from plants of the families Fabaceae (Leguminosae), Cruciferaceae, Chenopodiaceae, and Poaceae, such as soy bean meal, lupin meal and rapeseed meal.

In a particular embodiment, the vegetable protein source is material from one or more plants of the family Fabaceae, e.g. soybean, lupine, pea, or bean. In another particular embodiment, the vegetable protein source is material from one or more plants of the family Chenopodiaceae, e.g. beet, sugar beet, spinach or quinoa.

Other examples of vegetable protein sources are rapeseed, sunflower seed, cotton seed, cabbage and cereals such as barley, wheat, rye, oat, maize (corn), rice, triticale, and sorghum.

In still further particular embodiments, the animal feed composition of the invention contains 0-80% maize; and/or 0-80% sorghum; and/or 0-70% wheat; and/or 0-70% Barley; and/or 0-30% oats; and/or 0-30% rye; and/or 0-40% soybean meal; and/or 0-25% fish meal; and/or 0-25% meat and bone meal; and/or 0-20% whey.

Animal diets can e.g. be manufactured as mash feed (non pelleted) or pelleted feed. Typically, the milled feed-stuffs are mixed and sufficient amounts of essential vitamins and minerals are added according to the specifications for the species in question. The spore strain can be added as solid or liquid formulation. It is at present contemplated that the Bacillus strain is administered in one or more of the following amounts (dosage ranges): 10 E2-14, 10 E4-12, 10 E6-10, 10 E7-9, preferably 10 E8 CFU/g of feed (the designation E meaning exponent, viz., e.g., 10 E2-14 means 102-1014).

It is further an object of the invention to provide a viable or inert spore, wherein said spore is genetically modified with a genetic code comprising at least one genetic construct encoding an enzymatically active enzyme, a bioactive polypeptide, an affinity ligand or a immobilized protein as specified herein above and a genetic construct encoding a amino acid sequence of a spore-specific surface-enzyme.

According to a further aspect, the present invention provides B. subtilis strains transformed according to the inventions as defined above. B. subtilis strains are SD39, SD48, SD50; SD60, SD 130, SD 140 and SD 150 which derive form B. subtilis parent strain deposited under Bacillus Genetic Stock Center 1A747.

The present invention will now be illustrated in more detail by the following examples, which are not meant to limit the scope of the invention. These examples are described with reference to the drawing. In the drawing

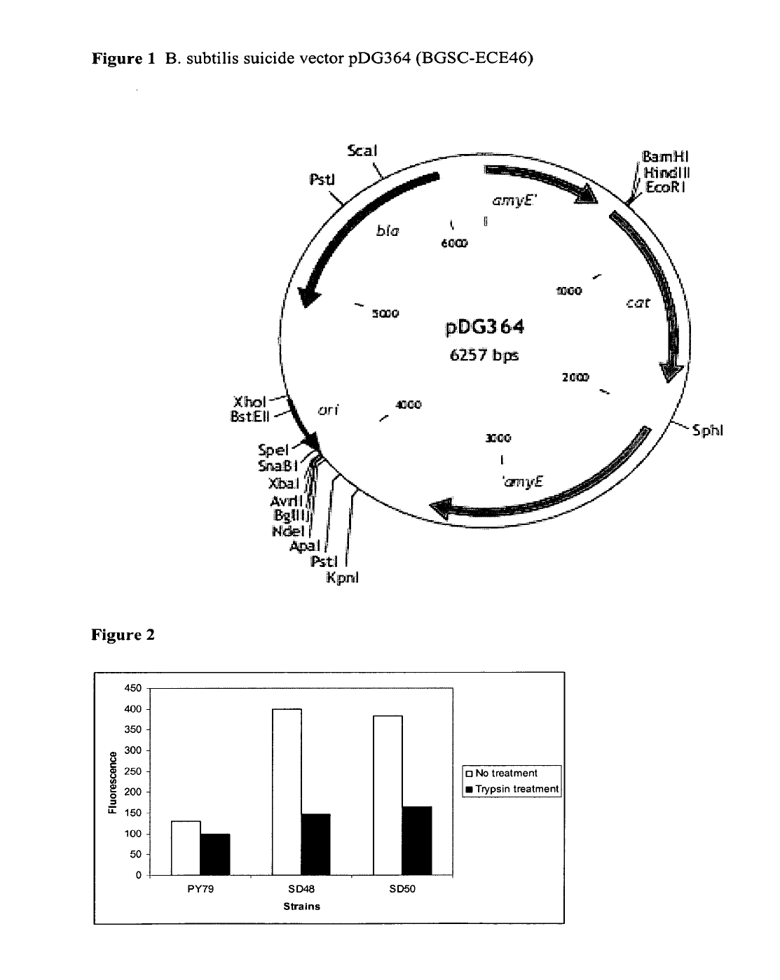

FIG. 1 shows a map of the B. subtilis vector pDG364,

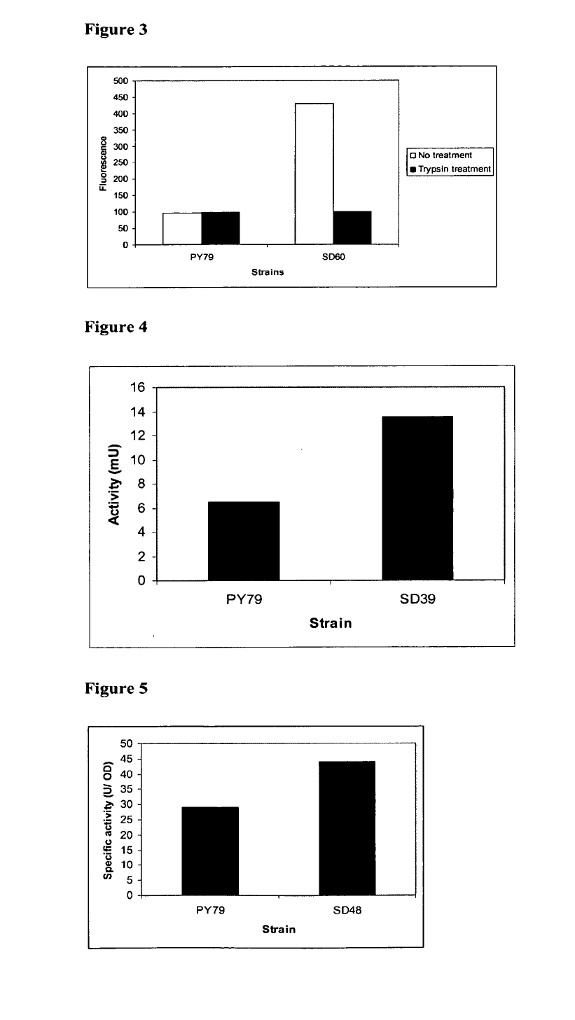

FIGS. 2 and 3 show intensity histograms of strains engineered according to example 5 and 6 compared to the wild type strains, and

FIGS. 4 to 6 show specific enzyme activities of strains engineered according to example 7, 8 or 9 compared to the wild type strains.

Applicant describes in the examples below the construction of a system aimed at the display of an enzymatic activity on the spore surface. Applicant has used the entire wild-type CotG protein as carrier and fused it, in frame, at the carboxyl-terminus end, with the gene encoding the phosphatase activity (Example 1). Significant phosphatase activity was found associated with engineered purified spore compared to non-engineered spores (Example 7). Equivalent constructions (translational C-terminus fusion to CotG), which have been designed to display phytase activity at the spore surface (B. subtilis endogenous phy activity) (Example 2), have also demonstrated specific enzymatic activity (Example 8). Instead of translational fusions to spore structural genes, passenger bioactive molecules (enzymes, bacteriocins, affinity ligands), can also be fused to spore-specific enzymes like oxdD or cotQ. Such a design is described in examples 3, where the phy gene is fused to the carboxyl-terminus of the oxalate decarboxylase encoded by oxdD (example 3) or in example 4 where the uidA gene encoding β-glucuronidase is fused to the carboxyl-terminal of oxdD. Specific display and corresponding enzymatic activities have been observed (examples 6 and 9 for oxdD-uidA). Display was also specifically demonstrated for cotG-phy and oxdD-phy fusions (example 5). Other example could use other enzyme-encoding genes like cotQ (encoding a reticuline oxidase-like protein) or cotA (encoding a laccase) as carriers. The main advantage of passenger fusions to carrier enzymes resides in the easy detection of the engineered fusion proteins, by straight-forward assaying the carrier enzymatic activity to demonstrate display, instead of time-consuming immuno-detection experiments that also requires expensive specific equipment. Another advantage of the enzymes can possibly be their easier amenability to overexpression than structural protein where stoiechiometric unbalance could lead to fragile spores.

### EXAMPLES

### General Methodology

In the first paragraphs the general methodology is summarized:

Strains and plasmids. Bacillus subtilis strains of the present invention are derived from strain 1A747 (Bacillus Genetic Stock Center, The Ohio State University, Columbus, Ohio 43210 USA), which is a prototrophic derivative of B. subtilis 168 (trpC2) (GenBank AL009126). The chloramphenicol-resistance gene (cat) cassette was obtained from plasmid pC194 (GeneBank M19465, Cat#1E17 Bacillus Genetic Stock Center, The Ohio State University, Columbus, Ohio 43210 USA).
Plasmid for integration

### Cassette for LFH-PCR

Media. Standard minimal medium (MM) for B. subtilis contains 1× Spizizen salts, 0.04% sodium glutamate, and 0.5% glucose. Standard solid complete medium is Tryptone Blood Agar Broth (TBAB, Difco). Standard liquid complete medium is Veal Infusion-Yeast Extract broth (VY). The compositions of these media are described below:
TBAB medium: 33 g Difco Tryptone Blood Agar Base (Catalog #0232), 1 L water. Autoclave.
VY medium: 25 g Difco Veal Infusion Broth (Catalog #0344), 5 g Difco Yeast Extract (Catalog #0127), 1 L water. Autoclave.
Minimal Medium (MM): 100 ml 10× Spizizen salts; 10 ml 50% glucose; 1 ml 40% sodium glutamate, qsp 1 L water.
10× Spizizen salts: 140 g K₂HPO₄; 20 g (NH₄)₂SO₄; 60 g KH₂PO₄; 10 g Na₃ citrate.2H₂O; 2 g MgSO₄.7H₂O; qsp 1 L with water.
10×VFB minimal medium (10×VFB MM): 2.5 g Na-glutamate; 15.7 g KH₂PO₄; 15.7 g K₂HPO₄; 27.4 g Na₂HPO₄.12H₂O; 40 g NH₄Cl; 1 g citric acid; 68 g (NH₄)₂SO₄; qsp 1 L water.
Trace elements solution: 1.4 g MnSO₄.H₂O; 0.4 g CoCl₂.6H₂O; 0.15 g (NH₄)₆Mo₇O₂₄.4H₂O; 0.1 g AlCl₃.6H₂O; 0.075 g CuCl₂.2H₂O; qsp 200 ml water.
Fe solution: 0.21 g FeSO₄.7H₂O; qsp 10 ml water.
CaCl₂ solution: 15.6 g CaCl₂.2H₂O; qsp 500 ml water.
Mg/Zn solution: 100 g MgSO₄.7H₂O; 0.4 g ZnSO₄.7H₂O; qsp 200 ml water.
VFB MM medium: 100 ml 10×VFB MM; 10 ml 50% glucose; 2 ml Trace elements solution; 2 ml Fe solution; 2 ml CaCl₂ solution; 2 ml Mg/Zn solution; 882 ml sterile distilled water.
Schaeffer sporulating medium: Bacto-nutrient broth 8 g; 10 ml 10% (w/v) KCl; 10 ml 1.2% (w/v) MgSO₄.7H₂O; 0.5 ml 1M NaOH; qsp 1 L. Add 1 ml 1M Ca(NO₃)₄; 1 ml 0.01 MnCl₂; 1 ml 1 mM FeSO₄.
Molecular and genetic techniques. Standard genetic and molecular biology techniques are generally known in the art and have been previously described. DNA transformation, and other standard B. subtilis genetic techniques are also generally known in the art and have been described previously (Harwood and Cutting, 1992).
Spore purification. Following incubation at 37° C. for 24 h, cultures were centrifuged at 7000 rpm for 10 min. After careful removal of the supernatant, pellets were re-suspended into cold H₂O and left 48 h at 4° C. to allow lysis of the remaining vegetative cells. The spores were then collected by another centrifugation of 10 min at 7000 rpm and re-suspended into 1 ml of 20% Gastrograffin (Schering). This solution was layered on top of 25 ml of 50% Gastrograffin and centrifuge for 20 min at 7000 rpm at 4° C. After careful removal of the layers of the Gastrograffin gradient, the pellet contains free spores. The pellets were subsequently washed twice in cold water to eliminate trace of Gastrograffin. Purified spores were re-suspended in cold water and kept frozen at −80° C. when needed.
Immunofluorescence detection. Custom anti-phytase rabbit-IgG (Eurogentec) was generated by immunizing rabbits with a mix of 2 synthetic phytase-specific peptides CAEPGGGSKGQVVDRA and CHKQVNPRKLKDRSDG) and used as primary antibody (Ab1). Goat anti rabbit-IgG coupled with FITC (Eurogentec) was used as secondary antibody (Ab2). Pictures are taken with Visitron Coolsnap camera and analysed with Metamorph software (Molecular Devices GmbH).

Practically, 20 uL of spore suspensions were resuspended in 500 uL PBS (no trypsin treatment) or in 400 uL PBS+100 uL Trypsin 0.5% solution (Amimed, Trypsin-EDTA PBS 0.5% 5-51K00-H), for a 0.1% final concentration (trypsin treatment was used to demonstrate specificity of display). Incubation was performed at 37° C. for 1 h with gentle agitation. Spores were then washed with 500 uL PBS-BSA 2% (3 times, 5 min, 8000 rpm), then incubated on ice for 30 min (blocking) in 500 uL PBS-BSA 2%. 2 uL Ab1 (1:1000) were added to the 500 uL suspensions, and incubated o/n, 4° C., on a rotating tube holder. The next day, spores were washed 3 times with 500 uL PBS-BSA2% 5 min, 8000 rpm and resuspended in 500 uL. 2 uL Ab2 (1:1000) were then added to the 500 uL, for 1 hour at RT, on a rotating tube holder (protected form light). Spores were finally washed in 500 uL PBS alone (4 times, 5 min, 8000 rpm). Spores were then resuspended in 30 uL PBS and 3 uL were mounted on a 2% agar layer slide, for microscopic observations (lens×100). Pictures were taken for white light (brightfield) and for green fluorescence (Ex=490 nm, Em=520 nm). Exposure time was 2100 ms for the fluorescent pictures. The green background was to reduced (scale=50% low) on an identical way for all fluorescent pictures. The fluorescence signal was assessed by measuring the pixel intensity using Metamorph 7.1.0.0 software (Molecular Devices GmbH).

Fluorescent detection of β-glucuronidase. In situ detection of β-glucuronidase activity was performed using a fluorogenic substrate ImaGene Green C12FDGlcU (Molecular Probes). This substrate was used on purified spores according to the indications of the manufacturer (Molecular Probes). Absorption and emission of the reaction product were respectively 495 and 518 nm. The fluorescence signal was assessed by measuring the pixel intensity using Metamorph 7.1.0.0 software (Molecular Devices).
β-glucuronidase (GUS) assay. Spores or cultures were first re-suspended in 800 uL of Z buffer (60 mM Na2HPO4.7H2O, 40 mM NaH2PO4, 10 mM KCl, 1 mM MgSO4.7H₂O, 50 mM β-mercaptoethanol, pH7). Solutions were then equilibrated 3 min at 30° C. before addition of 200 uL of pNPG (p-nitrophenyl-β-D-glucuronide 4 mg/ml). Incubation was performed at 30° C. until development of a yellow color. Reaction was then stopped with 500 uL Na2CO3 (1M), while reaction time (T) was recorded. Samples were then centrifuged for 3 min at 14000 rpm, and spectrophotometer measurement of the supernatants was performed at 420 nm. β-glucuronidase activity (Miller Units) was defined as (1000× Abs₄₂₀)/T(min)×Absₛₚₒᵣₑₛ×V(ml). Act=in Miller unit/ml spore suspension; V=1 ml (0.02 ml (spore suspension).
alkaline phosphatase assay. Based on the method described by Bessey, Lowry and Brock. (1967), B. subtilis alkaline phosphatase activity was colorimetrically measured using pNPP as substrate (para-nitrophenol phosphate, Fluka 71768). Specific conditions were an optimal pH at 9-10 and requirements for Mg and Zn. Measurements were made at 405 nm after incubation at 37° C. Activity unit were defined as amount of enzyme that catalyze the release of 1 micromole of para-nitrophenol per minute at 37° C.
phytase assay. The assay was run at pH7.4 and 37° C. which are optimal for B. subtilis phytase. In a first reaction, inorganic orthophosphate was liberated from phytase activity. This reaction was stopped after 30 min, before a second reaction was performed to measure the released Pi at 820 nm.
Activity assay: 300 μL f buffer B (Tris-HCl 100 mM pH 7.4, CaCl2 1 mM, sodium phytate 2 mM pH 7.4) were pre-warmed at 37° C. for 5 min. 75 μL of sample to assay (or controls) were then added before incubation for 30 min at 55° C. Reaction was stopped by adding 375 uL of TCA 15%. Samples underwent then a centrifugation 14000 rpm, 5 min, in order to harvest the spores, which would interfere with the Abs820 nm measurement (next step).
Photometric measurement of the released Pi (Alko method). 50 μL of the previous supernatants were diluted with water (total volume 500 uL). Then 500 uL of reagent C (1 vol. 10% ascorbic acid, 1 vol. 2.5% ammonium molybdate, 3 vol. 1M H2SO4) were added. Incubation was performed at 50° C. during 20 min. Absorbance of cooled samples was then read at 820n and compared to a standard curve which was made by measuring the Pi of dilutions 1000, 2000 and 4000 of a 90 mM KH2PO4 solution. Abs820 nm was read after 30 min incubation, 37° C. with 500 uL reagent C (added to 500 uL KH2PO4 dilutions).

### EXAMPLE 1

### Construction of B. subtilis Strain SD39 Designed to Display Alkaline Phosphatase Activity

This example describes the construction of B. subtilis strain SD39 designed to display alkaline phosphatase (PhoA) activity at the spore surface through fusion with the spore structural protein CotG.

Construction of the gene fusions were started by independent PCR amplifications of carrier and passenger fragments, subsequently combined by overlapping PCR to generate the translational fusions B. subtilis alkaline phosphatase (PhoA) was engineered without its signal peptide (1 to 41 AA). The absence of signal peptide is further denominated as“SPfree”. First, the 549-bp long carrier fragment of cotG (including 455-bp upstream of the ATG) was amplified from B. subtilis 1A747 chromosomal (wild type B. subtilis strain PY79) DNA in a 50 μl reaction volume containing 1 μl of 40 mM dNTP's, 5 μl of 10× buffer and 0.75 μl PCR enzyme (Herculase, Stratagene), 0.1 ug of template and primers cotG/for/BamHI and cotG/rev listed in Table 1. The PCR reaction was performed for 30 cycles using an annealing temperature of 53° C. Then, the 1356-bp long passenger phoA fragment was amplified from B. subtilis 1A747 chromosomal DNA in a 50 μl reaction volume containing 1 μl of 40 mM dNTP's, 5 μl of 10× buffer and 0.75 μl PCR enzyme (Herculase, Stratagene), 0.1 ug of template and primers cotG3′-ala15-phoA and phoA/rev/HindIII listed in Table 1. The PCR reaction was performed for 30 cycles using an annealing temperature of 53° C.

TABLE 1
Primers used to generate a
cotG-ala15-phoA translational fusion

| | Nucleotide sequence | SEQ ID |
|---|---|---|
| Name | (5′>3′) | NO: |
| cotG/for/BamHI | ATGCGGATCCCAGTGTCCC | 1 |
| | TAGCTCCGAG | |
| cotG/rev | TTTGTATTTCTTTTTGACT | 2 |
| | ACCCAGC | |
| cotG3′-ala15- | AAGAATACTGGAAAGACGG | 3 |
| phoA | CAATTGCTGGGTAGTCAAA | |
| | AAGAAATACAAGCAGCAGC | |
| | AGCAGCAGCAGCAGCAGCA | |
| | GCAGCAGCAGCAGCAGCAA | |
| | TGAAAAAAATGAGTTTGTT | |
| phoA/rev/HindIII | ATGCAAGCTTTTAAGAAAG | 4 |
| | TGCTTCCTTATTTATTC | |

Underlined sequences were overlapping sequences

Finally, assembly of the overlapping carrier and passenger fragments was made by a two-step PCR in which the first step used 0.1 μg of each purified overlapping fragments in a in a 50 μl reaction volume containing 1 μl of 40 mM dNTP's, 5 μl of 10× buffer and 0.75 μl PCR enzyme (Herculase, Stratagene). PCR reaction was performed for 30 cycles using an annealing temperature of 53° C. The second step was performed with the same conditions using 1 μl of the first reaction and cotG/for/BamHI and phoA/rev/HindIII as primers (Table 1).

The cote phoA translational fusion (Table 2) was then cloned between the BamHI and HindIII sites into a B. subtilis suicide vector (pDG364; BGSC-46; Karmazyn-Campelli et al., 1989; FIG. 1) for subsequent ectopic integration within the non-essential amyE locus.

TABLE 2
Sequence of the coG-(ala)15-phoA-SPfree
translational fusion (SEQ ID NO: 5). BamHI
and HindIII cloning sites are in bold underlined.
cotG gene coding sequence is in bold. phy gene
coding sequence is underlined. Spacer region
is in upper case font.

GGATCCCAGTGTCCCTAGCTCCGAGAAAAAATCCAGAGACAATTTGTT
TCTCATCAAGGAAGGGTCTTTATACTCCGCATTTAAGTGAATCTCTCG
CGCGCCGCGGAATGTTTTCGGCTGATAAAAGGAAATATGGTATGACTT
CTTTTTGAAGTCTCTGATATGTGATCCCCGATAAGCGATATCAATATC
CAGCCTTTTTTGATTTACCTTCATCACAGCTGGCACCGGATCATCGTC
CCATATATCCTTTTTTAATTCACGCAAGTCTTTTGGATGAACAAACAG
CTGATAAAGCGGTAAATTGGATTGATTCTTCATCCATAATCCTCCTTA
CAAATTTTAGGCTTTTATTTTTATAAGATCTCAGCGGAACACTTATAC
ACTTTTTAAAACCGCGCGTACTATGAGGGTAGTAAGGATCTTCATCCT
TAACATATTTTTAAAAGGAGGATTTCAAATTGGGCCACTATTCCCATT
CTGACATCGAAGAAGCGGTGAAATCCGCAAAAAAAGAAGGTTTAAAGG
ATTATTTATACCAAGAGCCTCATGGAAAAAAACGCAGTCATAAAAAGT
CGCACCGCACTCACAAAAAATCTCGCAGCCATAAAAAATCATACTGCT
CTCACAAAAAATCTCGCAGTCACAAAAAATCATTCTGTTCTCACAAAA
AATCTCGCAGCCACAAAAAATCATACTGCTCTCACAAGAAATCTCGCA
GCCACAAAAAATCGTACCGTTCTCACAAAAAATCTCGCAGCTATAAAA
AATCTTACCGTTCTTACAAAAAATCTCGTAGCTATAAAAAATCTTGCC
GTTCTTACAAAAAATCTCGCAGCTACAAAAAGTCTTACTGTTCTCACA
AGAAAAAATCTCGCAGCTATAAGAAGTCATGCCGCACACACAAAAAAT
CTTATCGTTCCCATAAGAAATACTACAAAAAACCGCACCACCACTGCG
ACGACTACAAAAGACACGATGATTATGACAGCAAAAAAGAATACTGGA
AAGACGGCAATTGCTGGGTAGTCAAAAAGAAATACAAAGCAGCAGCAG
CAGCAGCAGCAGCAGCAGCAGCAGCAGCAGCAATGAAAAAAATGAGTT
TGTTTCAAAATATGAAATCAAAACTTCTGCCAATCGCCGCTGTTTCTG
TCCTTACAGCTGGAATCTTTGCCGGAGCTGAGCTTCAGCAAACAGAAA
AGGCCAGCGCCAAAAAACAAGACAAAGCTGAGATCAGAAATGTCATTG
TGATGATAGGCGACGGCATGGGGACGCCTTACATAAGAGCCTACCGTT
CCATGAAAAATAACGGTGACACACCGAATAACCCGAAGTTAACAGAAT
TTGACCGGAACCTGACAGGCATGATGATGACGCATCCGGATGACCCTG
ACTATAATATTACAGATTCAGCAGCAGCCGGAACAGCATTAGCGACAG
GCGTTAAGACATATAACAATGCAATTGGCGTCGATAAAAACGGAAAAA
AAGTGAAATCTGTACTTGAAGAGGCCAAACAGCAAGGCAAGTCAACAG
GGCTTGTCGCCACGTCTGAAATTAACCACGCCACTCCAGCCGCATATG
GCGCCCACAATGAATCACGGAAAAACATGGACCAAATCGCCAACAGCT
ATATGGATGACAAGATAAAAGGCAAACATAAAATAGACGTGCTGCTCG
GCGGCGGAAAATCTTATTTTAACCGCAAGAACAGAAACTTGACAAAGG
AATTCAAACAAGCCGGCTACAGCTATGTGACAACTAAACAAGCATTGA
AAAAAAATAAAGATCAGCAGGTGCTCGGGCTTTTCGCAGATGGAGGGC
TTGCTAAAGCGCTCGACCGTGACAGTAAAACACCGTCTCTCAAAGACA
TGACGGTTTCAGCAATTGATCGCCTGAACCAAAATAAAAAAGGATTTT
TCTTGATGGTCGAAGGGAGCCAGATTGACTGGGCGGCCCATGACAATG
ATACAGTAGGAGCCATGAGCGAGGTTAAAGATTTTGAGCAGGCCTATA
AAGCCGCGATTGAATTTGCGAAAAAAGACAAACATACACTTGTGATTG
CAACTGCTGACCATACAACCGGCGGCTTTACCATTGGCGCAAACGGGG
AAAAGAATTGGCACGCAGAACCGATTCTCTCCGCTAAGAAAACACCTG
AATTCATGGCCAAAAAAATCAGTGAAGGCAAGCCGGTTAAAGATGTGC
TCGCCCGCTATGCCAATCTGAAAGTCACATCTGAAGAAATCAAAAGCG
TTGAAGCAGCTGCACAGGCTGACAAAAGCAAAGGGGCCTCCAAAGCCA
TCATCAAGATTTTTAATACCCGCTCCAACAGCGGATGGACGAGTACCG
ATCATACCGGCGAAGAAGTACCGGTATACGCGTACGGCCCCGGAAAAG
AAAAATTCCGCGGATTGATTAACAATACGGACCAGGCAAACATCATAT
TTAAGATTTTAAAAACTGGAAAATAAAAGCTT

The resulting plasmid was named pSD16. Subsequent sequencing of the translational fusion revealed that the ala spacer was made only of 14 residues.

Following linearization with XhoI restriction endonuclease, plasmid pSD16 was transformed into strain PY79, resulting by double-crossover recombination at the non-essential amyE locus, to B. subtilis spore display strain SD39.

### EXAMPLE 2

### Construction of B. subtilis Strain SD48 Designed to Display Phytase Activity

This example describes the construction of B. subtilis strain SD48 designed to display phytase (phy) activity at the spore surface through fusion with the spore structural protein CotG.

TABLE 3
Sequence of the cotG-(ala)15-phy-SPfree
translational fusion (SEQ ID NO: 6). BamHI
and HindIII cloning sites are in bold underlined.
cotG gene coding sequence is in bold. phy gene
coding sequence is underlined. Spacer region is
in upper case font.

GGATCCCAGTGTCCCTAGCTCCGAGAAAAAATCCAGAGACAATTTGTTT
CTCATCAAGGAAGGGTCTTTATACTCCGCATTTAAGTGAATCTCTCGCG
CGCCGCGGAATGTTTTCGGCTGATAAAAGGAAATATGGTATGACTTCTT
TTTGAAGTCTCTGATATGTGATCCCCGATAAGCGATATCAATATCCAGC
CTTTTTTGATTTACCTTCATCACAGCTGGCACCGGATCATCGTCCCATA
TATCCTTTTTTAATTCACGCAAGTCTTTTGGATGAACAAACAGCTGATA
AAGCGGTAAATTGGATTGATTCTTCATCCATAATCCTCCTTACAAATTT
TAGGCTTTTATTTTTATAAGATCTCAGCGGAACACTTATACACTTTTTA
AAACCGCGCGTACTATGAGGGTAGTAAGGATCTTCATCCTTAACATATT
TTTAAAAGGAGGATTTCAAATTGGGCCACTATTCCCATTCTGACATCGA
AGAAGCGGTGAAATCCGCAAAAAAAGAAGGTTTAAAGGATTATTTATAC
CAAGAGCCTCATGGAAAAAAACGCAGTCATAAAAAGTCGCACCGCACTC
ACAAAAAATCTCGCAGCCATAAAAAATCATACTGCTCTCACAAAAAATC
TCGCAGTCACAAAAAATCATTCTGTTCTCACAAAAAATCTCGCAGCCAC
AAAAAATCATACTGCTCTCACAAGAAATCTCGCAGCCACAAAAAATCGT
ACCGTTCTCACAAAAAATCTCGCAGCTATAAAAAATCTTACCGTTCTTA
CAAAAAATCTCGTAGCTATAAAAAATCTTGCCGTTCTTACAAAAAATCT
CGCAGCTACAAAAAGTCTTACTGTTCTCACAAGAAAAAATCTCGCAGCT
ATAAGAAGTCATGCCGCACACACAAAAAATCTTATCGTTCCCATAAGAA
ATACTACAAAAAACCGCACCACCACTGCGACGACTACAAAAGACACGAT
GATTATGACAGCAAAAAAGAATACTGGAAAGACGGCAATTGCTGGGTAG
TCAAAAAGAAATACAAAGCAGCAGCAGCAGCAGCAGCAGCAGCAGCAGC
AGCAGCAGCAGCAGTGAATGAGGAACATCATTTCAAAGTGACTGCACAC
ACGGAGACAGATCCGGTCGCATCTGGCGATGATGCAGCAGATGACCCGG
CCATTTGGGTTCATGAAAAACACCCGGAAAAAAGCAAGTTGATTACAAC
AAATAAGAAGTCAGGGCTCGTTGTGTATGATTTAGACGGAAAACAGCTT
CATTCTTATGAGTTTGGCAAGCTCAATAATGTCGATCTGCGCTATGATT
TTCCATTGAACGGCGAAAAAATTGATATTGCTGCCGCATCCAACCGGTC
CGAAGGAAAAAATACAATTGAAGTATATGCAATAGACGGGGATAAAGGA
AAATTGAAAAGCATTACAGATCCGAACCATCCTATTTCCACCAATATTT
CTGAGGTTTATGGATTCAGCTTGTATCACAGCCAGAAAACAGGAGCATT
TTACGCATTAGTGACAGGCAAACAAGGGGAATTTGAGCAGTATGAAATT
GTTGATGGTGGAAAGGGTTATGTAACAGGGAAAAAGGTGCGTGAATTTA
AGTTGAATTCTCAGACCGAAGGCCTTGTTGCGGATGATGAGTACGGAAA
CCTATACATAGCAGAGGAAGATGAGGCCATCTGGAAATTTAACGCTGAG
CCCGGCGGAGGGTCAAAGGGGCAGGTTGTTGACCGTGCGACAGGAGATC
ATTTGACAGCTGATATTGAAGGACTGACAATCTATTATGCACCAAATGG
CAAAGGATATCTCATGGCTTCAAGTCAAGGAAATAACAGCTATGCAATG
TATGAACGGCAGGGGAAAAATCGCTATGTAGCCAACTTTGAGATTACAG
ATGGCGAGAAGATAGACGGTACTAGTGACACGGATGGTATTGATGTTCT
CGGTTTCGGACTTGGCCCAAAATATCCGTACGGGATTTTTGTGGCGCAG
GACGGCGAAAATATTGATAACGGACAAGCCGTCAATCAAAATTTCAAAA
TTGTATCGTGGGAACAAATTGCACAGCATCTCGGCGAAATGCCTGATCT
TCATAAACAGGTAAATCCGAGGAAGCTGAAAGACCGTTCTGACGGCTAG
TAAAAGCTT

The cotG-ala15-phy-SPfree synthetic translational fusion was cloned between the BamHI and HindIII sites into a B. subtilis suicide vector (pDG364; BGSC-46; Karmazyn-Campelli et al., 1989; FIG. 1) for subsequent ectopic integration within the non-essential amyE locus. The resulting plasmid was named pSD21.

Following linearization with XhoI restriction endonuclease, plasmid pSD21 was transformed into strain PY79, leading, by double-crossover recombination at the non-essential amyE locus, to B. subtilis spore display strain SD48.

### EXAMPLE 3

### Construction of B. subtilis Strain SD50 Designed to Display Phytase Activity

This example describes the construction of B. subtilis strain SD50 designed to display endogenous phytase activity (phy) at the spore surface through fusion with the spore coat enzyme OxdD.

TABLE 4
Sequence of the oxdD-ala10(NheI)-phy synthetic
translational fusion (SEQ ID NO: 7). BamHI and
HindIII cloning sites are in bold underlined.
oxdD gene coding sequence is in bold. phy gene
coding sequence is underlined. Spacer region is
in lower case font. NheI restriction site in the
spacer is in lower case underlined fonts.

GGATCCCACAGGTGATGAAATGCCGGGTGGGGGACGCATGGAGGACCA
TATTTCCACCTTTGATTATATGCCTGAAGATGAAGTGATAGGTCATGA
TGTATTAGTAAAAGTGGAGTGGAGGACAGGCCAGAAAAAACAGACAGA
AGCAATCAAATTACATAAGAAGCCATGGTATAAAAAATAGTTTATTTG
ATGTATTTGTGATCACATTGGTGGTCACTTTTTTATTTGCGGATTCCT
AGGCACAGCAATCTAAGATTCTGCATAGGCTGAAATAAAATCTTGTTC
ATTTCTAAAACGAGGTGCATGCTGTTGGAACAACAACCAATCAATCAT
GAAGACAGAAACGTGCCGCAGCCTATTCGAAGTGATGGAGCTGGAGCT
ATTGATACAGGCCCGCGAAATATAATACGGGATATTCAAAATCCGAAT
ATATTTGTTCCGCCTGTTACAGATGAGGGTATGATTCCTAACTTGAGA
TTTTCATTCTCAGACGCTCCCATGAAATTAGATCACGGCGGCTGGTCA
AGAGAAATCACCGTAAGACAGCTTCCGATTTCGACTGCGATTGCAGGT
GTAAACATGAGCTTAACTGCGGGAGGCGTCCGCGAGCTTCATTGGCAT
AAGCAAGCGGAGTGGGCTTATATGCTTTTGGGACGGGCACGTATCACC
GCTGTTGACCAAGACGGACGAAATTTCATTGCTGATGTTGGTCCCGGC
GACCTTTGGTACTTCCCGGCAGGAATTCCGCATTCCATACAGGGATTG
GAACACTGCGAGTTTCTGCTCGTTTTCGATGATGGGAACTTTTCTGAG
TTTTCAACGTTAACCATTTCAGATTGGCTTGCACACACACCAAAAGAT
GTTCTGTCTGCAAATTTCGGTGTCCCGGAGAATGCTTTCAACTCTCTT
CCGTCTGAGCAAGTCTATATCTACCAAGGGAATGTGCCGGGATCAGTC
GCCAGTGAAGACATTCAGTCACCATATGGAAAAGTCCCCATGACCTTT
AAACACGAGCTGTTAAATCAACCCCCAATTCAAATGCCAGGGGGGAGT
GTACGTTCAGATTGAGCCTGGCGCGATGAGAGAGCTTCATTGGCATCC
CAATAGCGATGAGTGGCAATATTATCTAACAGGACAGGGACGAATGAC
GGTATTTATCGGAAATGGGACTGCCCGCACATTTGATTATAGAGCAGG
CGACGTTGGATACGTGCCTTCTAATGCCGGACACTATATACAAAACAC
TGGTACAGAAACATTATGGTTTTTAGAAATGTTCAAAAGTAACCGCTA
TGCAGATGTGTCACTCAATCAGTGGATGGCATTGACGCCTAAAGAATT
AGTACAAAGCAACTTGAATGCTGGATCAGTCATGCTTGATTCTCTGCG
CAAGAAGAAAGTGCCTGTTGTGAAATATCCCGGTACGgcagcagcagc
agctagcgcagcagcagcaGTGAATGAGGAACATCATTTCAAAGTGAC
TGCACACACGGAGACAGATCCGGTCGCATCTGGCGATGATGCAGCAGA
TGACCCGGCCATTTGGGTTCATGAAAAACACCCGGAAAAAAGCAAGTT
GATTACAACAAATAAGAAGTCAGGGCTCGTTGTGTATGATTTAGACGG
AAAACAGCTTCATTCTTATGAGTTTGGCAAGCTCAATAATGTCGATCT
GCGCTATGATTTTCCATTGAACGGCGAAAAAATTGATATTGCTGCCGC
ATCCAACCGGTCCGAAGGAAAAAATACAATTGAAGTATATGCAATAGA
CGGGGATAAAGGAAAATTGAAAAGCATTACAGATCCGAACCATCCTAT
TTCCACCAATATTTCTGAGGTTTATGGATTCAGCTTGTATCACAGCCA
GAAAACAGGAGCATTTTACGCATTAGTGACAGGCAAACAAGGGGAATT
TGAGCAGTATGAAATTGTTGATGGTGGAAAGGGTTATGTAACAGGGAA
AAAGGTGCGTGAATTTAAGTTGAATTCTCAGACCGAAGGCCTTGTTGC
GGATGATGAGTACGGAAACCTATACATAGCAGAGGAAGATGAGGCCAT
CTGGAAATTTAACGCTGAGCCCGGCGGAGGGTCAAAGGGGCAGGTTGT
TGACCGTGCGACAGGAGATCATTTGACAGCTGATATTGAAGGACTGAC
AATCTATTATGCACCAAATGGCAAAGGATATCTCATGGCTTCAAGTCA
AGGAAATAACAGCTATGCAATGTATGAACGGCAGGGGAAAAATCGCTA
TGTAGCCAACTTTGAGATTACAGATGGCGAGAAGATAGACGGTACTAG
TGACACGGATGGTATTGATGTTCTCGGTTTCGGACTTGGCCCAAAATA
TCCGTACGGGATTTTTGTGGCGCAGGACGGCGAAAATATTGATAACGG
ACAAGCCGTCAATCAAAATTTCAAAATTGTATCGTGGGAACAAATTGC
ACAGCATCTCGGCGAAATGCCTGATCTTCATAAACAGGTAAATCCGAG
GAAGCTGAAAGACCGTTCTGACGGCTAGTAAAAGCTT

The oxdD-ala10(NheI)-phy synthetic translational fusion was then cloned between the BamHI and HindIII sites into a B. subtilis suicide vector (pDG364; BGSC-46; Karmazyn-Campelli et al., 1989; FIG. 1) for subsequent ectopic integration within the non-essential amyE locus. The resulting plasmid was named pSD22.

Following linearization with XhoI restriction endonuclease, plasmid pSD22 was transformed into strain PY79, leading, by double-crossover recombination at the non-essential amyE locus, to B. subtilis spore display strain SD50.

### EXAMPLE 4

### Construction of B. subtilis Strain SD60 Designed to Display β-Glucuronidase Activity

This example describes the construction of B. subtilis strain SD60 designed to display β-glucuronidase (GUS encoded by uidA E. coli gene) activity at the spore surface through fusion with the spore enzyme protein OxdD.

TABLE 5
Sequence of the oxdD-ala10(Nhel)-uidA synthetic
translational fusion (SEQ ID NO: 8). BamHI and
Hindlll cloning sites are in bold underlined.
oxdD gene coding sequence is in bold. uidA gene
coding sequence is underlined. Spacer region is
in lower case font. NheI restriction site in the
spacer is in lower case underlined fonts.

GGATCCCACAGGTGATGAAATGCCGGGTGGGGGACGCATGGAGGACCA
TATTTCCACCTTTGATTATATGCCTGAAGATGAAGTGATAGGTCATGA
TGTATTAGTAAAAGTGGAGTGGAGGACAGGCCAGAAAAAACAGACAGA
AGCAATCAAATTACATAAGAAGCCATGGTATAAAAAATAGTTTATTTG
ATGTATTTGTGATCACATTGGTGGTCACTTTTTTATTTGCGGATTCCT
AGGCACAGCAATCTAAGATTCTGCATAGGCTGAAATAAAATCTTGTTC
ATTTCTAAAACGAGGTGCATGCTGTTGGAACAACAACCAATCAATCAT
GAAGACAGAAACGTGCCGCAGCCTATTCGAAGTGATGGAGCTGGAGCT
ATTGATACAGGCCCGCGAAATATAATACGGGATATTCAAAATCCGAAT
ATATTTGTTCCGCCTGTTACAGATGAGGGTATGATTCCTAACTTGAGA
TTTTCATTCTCAGACGCTCCCATGAAATTAGATCACGGCGGCTGGTCA
AGAGAAATCACCGTAAGACAGCTTCCGATTTCGACTGCGATTGCAGGT
GTAAACATGAGCTTAACTGCGGGAGGCGTCCGCGAGCTTCATTGGCAT
AAGCAAGCGGAGTGGGCTTATATGCTTTTGGGACGGGCACGTATCACC
GCTGTTGACCAAGACGGACGAAATTTCATTGCTGATGTTGGTCCCGGC
GACCTTTGGTACTTCCCGGCAGGAATTCCGCATTCCATACAGGGATTG
GAACACTGCGAGTTTCTGCTCGTTTTCGATGATGGGAACTTTTCTGAG
TTTTCAACGTTAACCATTTCAGATTGGCTTGCACACACACCAAAAGAT
GTTCTGTCTGCAAAVTTCGGTGTCCCGGAGAATGCTTTCAACTCTCTT
CCGTCTGAGCAAGTCTATATCTACCAAGGGAATGTGCCGGGATCAGTC
GCCAGTGAAGACATTCAGTCACCATATGGAAAAGTCCCCATGACCTTT
AAACACGAGCTGTTAAATCAACCCCCAATTCAAATGCCAGGGGGGAGT
GTACGAATTGTGGATTCTTCTAACTTCCCAATTTCAAAAACGATAGCC
GCTGCACPTGTTCAGATTGAGCCTGGCGCGATGAGAGAGCTTCATTGG
CATCCCAATAGCGATGAGTGGCAATATTATCTAACAGGACAGGGACGA
ATGACGGTATTTATCGGAAATGGGACTGCCCGCACATTTGATTATAGA
GCAGGCGACGTTGGATACGTGCCTTCTAATGCCGGACACTATATACAA
AACACTGGTACAGAAACATTATGGTTTTTAGAAATGTTCAAAAGTAAC
CGCTATGCAGATGTGTCACTCAATCAGTGGATGGCATTGACGCCTAAA
GAATTAGTACAAAGCAACTTGAATGCTGGATCAGTCATGCTTGATTCT
CTGCGCAAGAAGAAAGTGCCTGTTGTGAAATATCCCGGTACGgcagca
gcagctagcgcagcagcagcagcaATGTTACGTCCTGTAGAAACCCCA
ACCCGTGAAATCAAAAAACTCGACGGCCTGTGGGCATTCAGTCTGGAT
CGCGAAAACTGTGGAATTGATCAGCGTTGGTGGGAAAGCGCGTTACAA
GAAAGCCGGGCAATTGCTGTGCCAGGCAGTTTTAACGATCAGTTCGCC
GATGCAGATATTCGTAATTATGCGGGCAACGTCTGGTATCAGCGCGAA
GTCTTTATACCGAAAGGTTGGGCAGGCCAGCGTATCGTGCTGCGTTTC
GATGCGGTCACTCATTACGGCAAAGTGTGGGTCAATAATCAGGAAGTG
ATGGAGCATCAGGGCGGCTATACGCCATTTGAAGCCGATGTCACGCCG
TATGTTATTGCCGGGAAAAGTGTACGTATCACCGTTTGTGTGAACAAC
GAACTGAACTGGCAGACTATCCCGCCGGGAATGGTGATTACCGACGAA
AACGGCAAGAAAAAGCAGTCTTACTTCCATGATTTCTTTAACTATGCC
GGGATCCATCGCAGCGTAATGCTCTACACCACGCCGAACACCTGGGTG
GACGATATCACCGTGGTGACGCATGTCGCGCAAGACTGTAACCACGCG
TCTGTTGACTGGCAGGTGGTGGCCAATGGTGATGTCAGCGTTGAACTG
CGTGATGCGGATCAACAGGTGGTTGCAACTGGACAAGGCACTAGCGGG
ACTTTGCAAGTGGTGAATCCGCACCTCTGGCAACCGGGTGAAGGTTAT
CTCTATGAACTGTGCGTCACAGCCAAAAGCCAGACAGAGTGTGATATC
TACCCGCTTCGCGTCGGCATCCGGTCAGTGGCAGTGAAGGGCGAACAG
TTCCTGATTAACCACAAACCGTTCTACTTTACTGGCTTTGGTCGTCAT
GAAGATGCGGACTTGCGTGGCAAAGGATTCGATAACGTGCTGATGGTG
CACGACCACGCATTAATGGACTGGATTGGGGCCAACTCCTACCGTACC
TCGCATTACCCTTACGCTGAAGAGATGCTCGACTGGGCAGATGAACAT
GGCATCGTGGTGATTGATGAAACTGCTGCTGTCGGCTTTAACCTCTCT
TTAGGCATTGGTTTCGAAGCGGGCAACAAGCCGAAAGAACTGTACAGC
GAAGAGGCAGTCAACGGGGAAACTCAGCAAGCGCACTTACAGGCGATT
AAAGAGCTGATAGCGCGTGACAAAAACCACCCAAGCGTGGTGATGTGG
AGTATTGCCAACGAACCGGATACCCGTCCGCAAGGTGCACGGGAATAT
TTCGCGCCACTGGCGGAAGCAACGCGTAAACTCGACCCGACGCGTCCG
ATCACCTGCGTCAATGTAATGTTCTGCGACGCTCACACCGATACCATC
AGCGATCTCTTTGATGTGCTGTGCCTGAACCGTTATTACGGATGGTAT
GTCCAAAGCGGCGATTTGGAAACGGCAGAGAAGGTACTGGAAAAAGAA
CTTCTGGCCTGGCAGGAGAAACTGCATCAGCCGATTATCATCACCGAA
TACGGCGTGGATACGTTAGCCGGGCTGCACTCAATGTACACCGACATG
TGGAGTGAAGAGTATCAGTGTGCATGGCTGGATATGTATCACCGCGTC
TTTGATCGCGTCAGCGCCGTCGTCGGTGAACAGGTATGGAATTTCGCC
GATTTTGCGACCTCGCAAGGCATATTGCGCGTTGGCGGTAACAAGAAA
GGGATCTTCACTCGCGACCGCAAACCGAAGTCGGCGGCTTTTCTGCTG
CAAAAACGCTGGACTGGCATGAACTTCGGTGAAAAACCGCAGCAGGGA
GGCAAACAATGAtaaAAGCTT

After PCR amplification the uidA gene was inserted between NheI and HindIII sites of vector pSD22 at the 3′-end of the oxdD open reading frame, generating a oxdD-ala10-uidA translational fusion for subsequent ectopic integration within the non-essential amyE locus. The resulting plasmid was named pSD27.

Following linearization with XhoI restriction endonuclease, plasmid pSD27 was transformed into strain PY79, leading, by double-crossover recombination at the non-essential amyE locus, to B. subtilis spore display strain SD60.

### EXAMPLE 5

### Specific Display of Phytase Enzyme Associated to Spores Surface Using Two Kinds of Carriers

This example demonstrates that phytase enzyme is specifically displayed at the spore surface of cotG-engineered strain SD48 and oxdD-engineered strain SD50.

Using the immuno-detection procedure described in the general methodology section, phytase-specific higher fluorescence intensity was observed for spores of strains SD48 and SD50, than with PY79 spores (FIG. 2). Fluorescence signals of these two strains dropped significantly when the spores underwent a trypsin digestion of the displayed fusions.

FIG. 2: Fluorescence intensity histograms of strain SD48 and SD50 compared to wild type strain PY79. Empty bars represent fluorescence of spores that have not undergone trypsin treatment. Black bars represent fluorescence activities of spore treated with protease. The fluorescence signal is an average of the pixel intensity in spores, measured by Metamorph software. SD48 contains a cotG-(ala)15-phy-SP free translational fusion; SD50 contains a oxdD-ala10(NheI)-phy-SP free translational fusion.

In conclusion, immuno-detection by microscopy demonstrated evidence that two kinds of carriers can successfully display B. subtilis phytase at the spore surface, coat structural proteins (like CotG) and but also spore associated enzymes, like OxdD.

### EXAMPLE 6

### Display of β-Glucuronidase Associated to Spores from oxdD-Engineered Strain SD60

This example demonstrates that β-glucuronidase enzyme is associated with spores from oxdD-engineered strain SD60 and displayed at its surface.

A different technology based on specific modification of the fluorogenic substrate ImaGene Green C12FDGlcU (Molecular Probes) has been used in this experiment to demonstrate the display of an active enzyme using spore specific enzyme carrier OxdD (FIG. 3).

FIG. 3: Fluorescence intensity histograms of strain SD60 compared to wild type strain PY79. Empty bars represent fluorescence of spores that have not undergone trypsin treatment. Black bars represent fluorescence activities of spore treated with protease. The fluorescence signal is an average of the pixel intensity in spores, measured by Metamorph software. SD60 contains a oxdD-ala10-uidA translational fusion.

In conclusion, trypsin treatment demonstrated the specific display of the β-glucuronidase at the spore surface using a spore associated enzyme, like OxdD, as carrier.

### EXAMPLE 7

### Phosphatase Activity Associated to Spores from cotG-Engineered Strain SD39

This example demonstrates that phosphatase enzymatic activity is associated with spores from cotG-engineered strain SD39.

Alkaline phospahatase enzymatic activity was measured on pure spore engineered to display the passenger enzyme with the core structural protein CotG (FIG. 4).

FIG. 4: Alkaline phosphatase activity associated to SD39 pure spore solution using colorimetric assay. Control strain was wild type strain PY79. Activities are in mUnits.

### EXAMPLE 8

### Phytase Activity Associated to Spores from cotG-Engineered Strain SD48

This example demonstrates that phytase enzymatic activity is associated with spores from cotG-engineered strain SD48 (FIG. 5).

FIG. 5: Phytase phosphatase activity associated to SD48 pure spore solution using colorimetric assay. Control strain was wild type strain PY79. Specific activities are in Units/Optical Density 580 nm.

### EXAMPLE 9

### β-Glucuronidase Activity Associated to Spores from oxdD-Engineered Strain SD60

This example demonstrates that β-glucuronidase enzymatic activity is associated with spores from oxdD-engineered strain SD60 and specifically displayed at its surface.

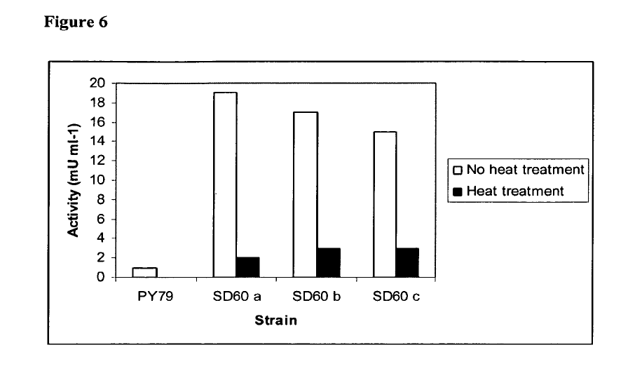

Based on classical colorimetric assay using pNPG as substrate (reading at 420 nm), β-glucuronidase activity was assessed in triplicate on SD60 pure spores prepared as described earlier (FIG. 6). Heat treatment was performed to denature enzymes and demonstrate specificity of the reported activity.

FIG. 6: β-glucuronidase activity of SD60 pure spore using colorimetric assay based on pNPG. Strain SD60 was tested in triplicates a, b, c. Empty bars represent enzymatic activity on pure spores. Black bars represent activities of pure spores heated during 15 min at 60° C. before performing the colorimetric enzymatic assay. SD60 contains an oxdD-ala10-uidA translational fusion. Control strain was wild type strain PY79. Activities are in Miller units.

In conclusion, this example demonstrates specific reporter enzymatic activity at the spore surface of a strain engineered to display enzyme through translational fusion to spore associated enzymes.

### EXAMPLE 10

### Display of Affinity Ligands

Display of affinity ligands at the spore surface in order to capture biomolecules is described in this example. The Aspergillus niger pex5 gene encodes for a protein which is recognizing specifically PTS-1 motifs [e.g. SKL (serine-lysine-leucine) motifs or PRL (proline-arginine-leucine)]. The PTS-1 motif can be engineered at the carboxyl-terminal of protein for specific tagging and subsequent capture of the tagged protein. This example describes the construction of B. subtilis strain SD 130 designed to display A. niger Pex5 PTS-1-affine protein at the spore surface through fusion with the spore coat protein CotC.

TABLE 6
Sequence of the cotC-ala10-pex5 translational
fusion (SEQ ID NO: 9). BamHI and HindIII cloning
sites are in bold underlined. cotC gene coding
sequence is in bold. pex5 gene coding sequence is
underlined. Spacer region is in lower case font.

GGATCCTTATTTTGTTTGTGGGTTTTTTAGTATTTGGGCCTGATAAAC
TGCCGGCGCTTGGCCGTGCAGCAGGAAAAGCCTTATCAGAATTTAAAC
AAGCAACAAGCGGACTGACTCAGGATATCAGAAAAAATGACTCAGAAA
ACAAAGAAGACAAACAAATGTAGGATAAATCGTTTGGGCCGATGAAAA
ATCGGCTCTTTATTTTGATTTGTTTTTGTGTCATCTGTCTTTTTCTAT
CATTTGGACAGCCCTTTTTTCCTTCTATGATTTTAACTGTCCAAGCCG
CAAAATCTACTCGCCGTATAATAAAGCGTAGTAAAAATAAAGGAGGAG
TATATATGGGTTATTACAAAAAATACAAAGAAGAGTATTATACGGTCA
AAAAAACGTATTATAAGAAGTATTACGAATATGATAAAAAAGATTATG
ACTGTGATTACGACAAAAAATATGATGACTATGATAAAAAATATTATG
ATCACGATAAAAAAGACTATGATTATGTTGTAGAGTATAAAAAGCATA
AAAAACACTACgcagcagcagcagcagcagcagcagcagcaATGTCCT
TCCTTGGTGGCGCCGAGTGCTCGACGGCGGGCAATCCGTTGACTCAGT
TCACCAAGCACGTCCAAGATGATAAGTCCCTACAGAGAGATCGCCTCG
TGGGGCGAGGCCCAGGAGGCATGCAAGAAGGCATGCGGTCCCGGGGTA
TGATGGGAGGACAAGATCAGATGATGGACGAATTCGCCCAACAACCCG
GCCAGATCCCCGGTGCTCCCCCGCAACCGTTCGCTATGGAACAGCTGC
GACGCGAGCTAGATCAGTTCCAAACCACACCTCCGAGGACGGGCTCCC
CCGGCTGGGCGGCCGAGTTCGATGCGGGCGAGCATGCCCGGATGGAGG
CTGCGTTTGCCGGGCCCCAGGGCCCCATGATGAATAATGCGTCGGGAT
TTACGCCCGCGGAGTTTGCCCGGTTCCAGCAGCAGAGTCGGGCTGGCA
TGCCTCAGACGGCTAACCATGTGGCGTCTGCCCCGTCGCCGATGATGG
CTGGGTACCAGCGGCCCATGGGTATGGGAGGGTATATGGGCATGGGTG
GAATGGGGATGATGCCGCAGACATTTAACCCGATGGCGATGCAGCAGC
AGCCGGCAGAGGCGACTACGCAGGACAAGGGCAAGGGACGCATGGTGG
AGCTGGACGACGAGAACTGGGAGGCACAGTTTGCCGAGATGGAGACGG
CGGATACCCAGAAATTGGACGATGAGGCCAACGCAGCTGTGGAGGCAG
AGCTGAATGATCTGGATAGGTCAGTCCCCCAAGATTCGGGCGATAGTG
CCTTTGAAAGCGTGTGGCAACGGGTCCAAGCTGAGACCGCAACAAACA
GGAAACTGGCCGAGGGCGAGACCGACTTTAACATTGACGACAATCTGC
ATATGGGTGAGATGGGCGAATGGGACGGATTCGATACGCTTAACACGC
GCTTCCGGAACCCTCAACTAGGCGATTATATGTTCGAAGAAGATAACG
TGTTCCGGAGCGTGAGCAATCCTTTCGAAGAGGGAGTGAAGATCATGC
GCGAGGGTGGAAACCTCTCCCTGGCTGCCTTGGCTTTCGAGGCGGCAG
TCCAGAAAGATCCTCAACATGTCCAGGCCTGGACCATGCTGGGATCGG
CTCAGGCGCAGAACGAGAAGGAGCTTCCCGCCATCAGAGCGCTGGAGC
AGGCACTTAAGATTGATGCTAACAATCTGGATGCGCTGATGGGACTGG
CTGTTTCCTACACCAACGAGGGCTATGACTCGACATCGTACCGCACTT
TGGAGCGTTGGCTGTCAGTCAAGTACCCCCAGATTATCAACCCTAATG
ATGTTTCATCGGAAGCCGACTTGGGCTTTACGGACCGCCAGCTCCTGC
ACGACCGTGTCACCGATCTCTTCATCCAGGCTGCTCAGCTGTCGCCAT
CTGGCGAGCAAATGGACCCGGACGTCCAGGTCGGTCTTGGCGTTCTCT
TCTACTGCGCAGAGGAGTATGACAAGGCGGTCGATTGCTTCTCTGCTG
CGTTGGCGTCCACGGAATCCGGAACGTCGAACCAACAGGAGCAGCTCC
ACCTGCTGTGGAACCGTCTGGGTGCTACGCTTGCCAACTCGGGTCGCT
CCGAGGAGGCGATCGAGGCCTACGAGCAGGCGCTGAACATCAATCCCA
ACTTCGTCCGGGCACGGTACAACCTGGGTGTGTCGTGCATCAACATCG
GCTGCTACCCAGAAGCCGCGCAACACCTGCTGGGAGCGCTATCGATGC
ACCGGGTGGTTGAGCAGGAAGGTCGAGAGCGGGCACGTGAGATTGTTG
GGGGCGAGGGTGGCATTGACGACGAGCAGCTGGATCGCATGATTCATG
TCAGCCAGAATCAGAGTACCAACCTGTACGACACGTTGCGGCGAGTAT
TTAGCCAGATGGGACGACGCGATCTGGCTGATCAGGTGGTGGCGGGGA
TGGATGTCAATGTGTTCCGACGGGAGTTTGAGTTCTAATAAAAGCTT

The cotC-ala10-pex5 translational fusion was then cloned between the BamHI and HindIII sites into a B. subtilis suicide vector (pDG364; BGSC-46; Karmazyn-Campelli et al., 1989; FIG. 1) for subsequent ectopic integration within the non-essential amyE locus. The resulting plasmid was named pSD130.

Following linearization with XhoI restriction endonuclease, plasmid pSD130 was transformed into B. subtilis wild type strain PY79, generating, by double-crossover recombination at the non-essential amyE locus, B. subtilis spore display strain SD130.

### EXAMPLE 11

### Construction of B. subtilis Strain SD140 Designed to Display A. niger PTS-1-Affine Pex5 Protein

This example describes the construction of B. subtilis strain SD 140 designed to display A. niger PTS-1-affine pex5 protein at the spore surface through fusion with the spore coat enzyme OxdD.

TABLE 7
Sequence of the oxdD-ala10(NheI)-pex5 synthetic
translational fusion (SEQ ID NO: 10). BamHI and
HindIII cloning sites are in bold underlined.
oxdD gene coding sequence is in bold. pex5 gene
coding sequence is underlined. Spacer region is
in lower case font. NheI restriction site in the
spacer is in lower case underlined fonts.

GGATCCCACAGGTGATGAAATGCCGGGTGGGGGACGCATGGAGGACCA
TATTTCCACCTTTGATTATATGCCTGAAGATGAAGTGATAGGTCATGA
TGTATTAGTAAAAGTGGAGTGGAGGACAGGCCAGAAAAAACAGACAGA
AGCAATCAAATTACATAAGAAGCCATGGTATAAAAAATAGTTTATTTG
ATGTATTTGTGATCACATTGGTGGTCACTTTTTTATTTGCGGATTCCT
AGGCACAGCAATCTAAGATTCTGCATAGGCTGAAATAAAATCTTGTTC
ATTTCTAAAACGAGGTGCATGCTGTTGGAACAACAACCAATCAATCAT
GAAGACAGAAACGTGCCGCAGCCTATTCGAAGTGATGGAGCTGGAGCT
ATTGATACAGGCCCGCGAAATATAATACGGGATATTCAAAATCCGAAT
ATATTTGTTCCGCCTGTTACAGATGAGGGTATGATTCCTAACTTGAGA
TTTTCATTCTCAGACGCTCCCATGAAATTAGATCACGGCGGCTGGTCA
AGAGAAATCACCGTAAGACAGCTTCCGATTTCGACTGCGATTGCAGGT
GTAAACATGAGCTTAACTGCGGGAGGCGTCCGCGAGCTTCATTGGCAT
AAGCAAGCGGAGTGGGCTTATATGCTTTTGGGACGGGCACGTATCACC
GCTGTTGACCAAGACGGACGAAATTTCATTGCTGATGTTGGTCCCGGC
GACCTTTGGTACTTCCCGGCAGGAATTCCGCATTCCATACAGGGATTG
GAACACTGCGAGTTTCTGCTCGTTTTCGATGATGGGAACTTTTCTGAG
TTTTCAACGTTAACCATTTCAGATTGGCTTGCACACACACCAAAAGAT
GTTCTGTCTGCAAATTTCGGTGTCCCGGAGAATGCTTTCAACTCTCTT
CCGTCTGAGCAAGTCTATATCTACCAAGGGAATGTGCCGGGATCAGTC
GCCAGTGAAGACATTCAGTCACCATATGGAAAAGTCCCCATGACCTTT
AAACACGAGCTGTTAAATCAACCCCCAATTCAAATGCCAGGGGGGAGT
GTACGTTCAGATTGAGCCTGGCGCGATGAGAGAGCTTCATTGGCATCC
CAATAGCGATGAGTGGCAATATTATCTAACAGGACAGGGACGAATGAC
GGTATTTATCGGAAATGGGACTGCCCGCACATTTGATTATAGAGCAGG
CGACGTTGGATACGTGCCTTCTAATGCCGGACACTATATACAAAACAC
TGGTACAGAAACATTATGGTTTTTAGAAATGTTCAAAAGTAACCGCTA
TGCAGATGTGTCACTCAATCAGTGGATGGCATTGACGCCTAAAGAATT
AGTACAAAGCAACTTGAATGCTGGATCAGTCATGCTTGATTCTCTGCG
CAAGAAGAAAGTGCCTGTTGTGAAATATCCCGGTACGgcagcagcagc
agctagcgcagcagcagcaATGTCCTTCCTTGGTGGCGCCGAGTGCTC
GACGGCGGGCAATCCGTTGACTCAGTTCACCAAGCACGTCCAAGATGA
TAAGTCCCTACAGAGAGATCGCCTCGTGGGGCGAGGCCCAGGAGGCAT
GCAAGAAGGCATGCGGTCCCGGGGTATGATGGGAGGACAAGATCAGAT
GATGGACGAATTCGCCCAACAACCCGGCCAGATCCCCGGTGCTCCCCC
GCAACCGTTCGCTATGGAACAGCTGCGACGCGAGCTAGATCAGTTCCA
AACCACACCTCCGAGGACGGGCTCCCCCGGCTGGGCGGCCGAGTTCGA
TGCGGGCGAGCATGCCCGGATGGAGGCTGCGTTTGCCGGGCCCCAGGG
CCCCATGATGAATAATGCGTCGGGATTTACGCCCGCGGAGTTTGCCCG
GTTCCAGCAGCAGAGTCGGGCTGGCATGCCTCAGACGGCTAACCATGT
GGCGTCTGCCCCGTCGCCGATGATGGCTGGGTACCAGCGGCCCATGGG
TATGGGAGGGTATATGGGCATGGGTGGAATGGGGATGATGCCGCAGAC
ATTTAACCCGATGGCGATGCAGCAGCAGCCGGCAGAGGCGACTACGCA
GGACAAGGGCAAGGGACGCATGGTGGAGCTGGACGACGAGAACTGGGA
GGCACAGTTTGCCGAGATGGAGACGGCGGATACCCAGAAATTGGACGA
TGAGGCCAACGCAGCTGTGGAGGCAGAGCTGAATGATCTGGATAGGTC
AGTCCCCCAAGATTCGGGCGATAGTGCCTTTGAAAGCGTGTGGCAACG
GGTCCAAGCTGAGACCGCAACAAACAGGAAACTGGCCGAGGGCGAGAC
CGACTTTAACATTGACGACAATCTGCATATGGGTGAGATGGGCGAATG
GGACGGATTCGATACGCTTAACACGCGCTTCCGGAACCCTCAACTAGG
CGATTATATGTTCGAAGAAGATAACGTGTTCCGGAGCGTGAGCAATCC
TTTCGAAGAGGGAGTGAAGATCATGCGCGAGGGTGGAAACCTCTCCCT
GGCTGCCTTGGCTTTCGAGGCGGCAGTCCAGAAAGATCCTCAACATGT
CCAGGCCTGGACCATGCTGGGATCGGCTCAGGCGCAGAACGAGAAGGA
GCTTCCCGCCATCAGAGCGCTGGAGCAGGCACTTAAGATTGATGCTAA
CAATCTGGATGCGCTGATGGGACTGGCTGTTTCCTACACCAACGAGGG
CTATGACTCGACATCGTACCGCACTTTGGAGCGTTGGCTGTCAGTCAA
GTACCCCCAGATTATCAACCCTAATGATGTTTCATCGGAAGCCGACTT
GGGCTTTACGGACCGCCAGCTCCTGCACGACCGTGTCACCGATCTCTT
CATCCAGGCTGCTCAGCTGTCGCCATCTGGCGAGCAAATGGACCCGGA
CGTCCAGGTCGGTCTTGGCGTTCTCTTCTACTGCGCAGAGGAGTATGA
CAAGGCGGTCGATTGCTTCTCTGCTGCGTTGGCGTCCACGGAATCCGG
AACGTCGAACCAACAGGAGCAGCTCCACCTGCTGTGGAACCGTCTGGG
TGCTACGCTTGCCAACTCGGGTCGCTCCGAGGAGGCGATCGAGGCCTA
CGAGCAGGCGCTGAACATCAATCCCAACTTCGTCCGGGCACGGTACAA
CCTGGGTGTGTCGTGCATCAACATCGGCTGCTACCCAGAAGCCGCGCA
ACACCTGCTGGGAGCGCTATCGATGCACCGGGTGGTTGAGCAGGAAGG
TCGAGAGCGGGCACGTGAGATTGTTGGGGGCGAGGGTGGCATTGACGA
CGAGCAGCTGGATCGCATGATTCATGTCAGCCAGAATCAGAGTACCAA
CCTGTACGACACGTTGCGGCGAGTATTTAGCCAGATGGGACGACGCGA
TCTGGCTGATCAGGTGGTGGCGGGGATGGATGTCAATGTGTTCCGACG
GGAGTTTGAGTTCTAATAAAAGCTT

The oxdD-ala10(NheI)-pex5 synthetic translational fusion was then cloned between the BamHI and HindIII sites into a B. subtilis suicide vector (pDG364; BGSC-46; Karmazyn-Campelli et al., 1989; FIG. 1) for subsequent ectopic integration within the non-essential amyE locus. The resulting plasmid was named pSD140.

Following linearization with XhoI restriction endonuclease, plasmid pSD140 was transformed into wild type B. subtilis strain PY79, generating, by double-crossover recombination at the non-essential amyE locus, to B. subtilis spore display strain SD140.

In order to improve expression, and therefore the display of the heterologous passenger without modifying the amino acid sequence, the A. niger pex5 coding sequence (passenger sequence, underlined in Table 7) was codon-adapted for expression in B. subtilis. The relevant optimized passenger sequence, which was designed to be free of BamHI, HindIII and NheI sites, is detailed in Table 8 and strictly encodes the same protein that the passenger sequence of Table 7 (Table 9). The oxdD-ala10(NheI)-optipex5 synthetic translational fusion was subsequently cloned between the BamHI and HindIII sites into the B. subtilis suicide vector pDG364 (BGSC-46; Karmazyn-Campelli et al., 1989; FIG. 1) for ectopic integration within the non-essential amyE locus. The resulting plasmid was named pSD150. The recombinant strain obtained after transformation into PY79 was named SD150.

TABLE 8
Sequence of A.niger pex5 coding sequence
(underlined in Table 7), codon-adapted for
expression in B.subtilis. Underlined TAATAA
are stop codons: (SEQ ID NO: 11)

ATGTCTTTCCTTGGCGGTGCTGAGTGCTCAACTGCCGGAAACCCGCT
GACTCAATTCACAAAGCACGTTCAGGATGACAAATCACTTCAGCGTG
ACCGTCTTGTCGGACGCGGACCGGGCGGTATGCAGGAAGGCATGCGT
TCTCGCGGTATGATGGGCGGACAGGATCAAATGATGGATGAATTCGC
ACAGCAGCCAGGTCAAATCCCAGGTGCGCCGCCTCAGCCATTTGCGA
TGGAGCAGCTTCGCCGTGAGCTTGATCAATTCCAAACAACTCCACCT
CGTACTGGTTCTCCAGGCTGGGCAGCTGAATTCGACGCTGGTGAGCA
CGCCCGTATGGAAGCTGCTTTCGCCGGACCGCAAGGTCCAATGATGA
ACAACGCTTCAGGCTTCACTCCAGCTGAATTCGCCCGTTTCCAGCAG
CAGTCTCGTGCGGGTATGCCTCAAACGGCAAACCACGTTGCAAGTGC
TCCTTCTCCAATGATGGCTGGTTATCAGCGTCCGATGGGTATGGGCG
GATACATGGGTATGGGCGGTATGGGTATGATGCCTCAAACGTTCAAC
CCAATGGCGATGCAGCAGCAGCCTGCTGAAGCAACAACTCAAGACAA
AGGTAAAGGCCGTATGGTTGAGCTTGATGACGAAAACTGGGAAGCTC
AATTCGCTGAAATGGAAACTGCTGACACTCAAAAGCTAGATGATGAA
GCAAACGCTGCTGTTGAAGCTGAGCTGAACGATCTTGACCGTTCTGT
TCCTCAGGATTCAGGTGACAGTGCGTTTGAATCTGTTTGGCAGCGTG
TTCAGGCTGAAACTGCAACAAACCGCAAGCTGGCTGAAGGTGAAACT
GACTTCAACATCGATGACAACCTTCACATGGGTGAAATGGGTGAGTG
GGACGGTTTCGACACTTTAAACACTCGTTTCCGCAACCCTCAGCTTG
GTGATTACATGTTCGAAGAAGACAACGTATTCCGTTCTGTATCAAAC
CCATTTGAAGAAGGCGTAAAAATCATGCGTGAAGGCGGAAACCTTTC
TCTTGCTGCGCTTGCGTTTGAAGCTGCTGTTCAAAAAGACCCTCAGC
ACGTTCAGGCTTGGACGATGCTTGGTTCTGCTCAAGCTCAAAACGAA
AAAGAGCTTCCTGCCATCCGTGCGCTTGAGCAGGCTTTAAAAATCGA
TGCTAACAACCTTGATGCTTTAATGGGTCTTGCTGTCAGCTACACAA
ATGAAGGCTATGACAGCACTTCTTACCGTACGCTTGAGCGCTGGCTT
TCTGTAAAATACCCTCAAATCATCAACCCAAACGATGTATCAAGTGA
AGCTGATCTTGGCTTCACTGACCGTCAATTGCTTCATGACCGTGTAA
CTGATTTGTTCATTCAAGCTGCACAGCTTTCTCCATCTGGTGAGCAA
ATGGACCCTGATGTTCAAGTAGGTCTTGGTGTACTATTCTACTGTGC
TGAAGAATACGATAAAGCGGTTGACTGCTTCTCTGCTGCTCTTGCTT
CAACTGAAAGCGGAACTTCAAACCAGCAAGAGCAGCTTCATTTGCTA
TGGAACCGTCTTGGTGCGACGCTTGCAAACAGCGGACGCAGTGAAGA
AGCGATCGAAGCATATGAGCAGGCGCTGAACATCAACCCAAACTTCG
TTCGTGCGCGTTACAACCTAGGTGTATCTTGTATCAACATCGGCTGT
TATCCTGAAGCGGCACAGCATTTGCTTGGTGCTTTATCAATGCACCG
TGTTGTTGAGCAGGAAGGCCGTGAGCGTGCGCGTGAAATCGTCGGCG
GTGAAGGCGGTATCGATGATGAGCAGCTTGACCGCATGATTCACGTT
TCTCAAAACCAATCTACAAACCTATATGATACGCTTCGCCGTGTATT
CTCTCAAATGGGCAGAAGAGATCTTGCTGATCAGGTTGTAGCGGGTA
TGGATGTAAACGTATTCCGTCGTGAGTTTGAATTCTAATAA

TABLE 9
Amino acid sequence of the A. niger Pex5
protein (SEQ ID NO: 12).

MSFLGGAECSTAGNPLTQFTKHVQDDKSLQRDRLVGRGPGGMQEGMR
SRGMMGGQDQMMDEFAQQPGQIPGAPPQPFAMEQLRRELDQFQTTPP
RTGSPGWAAEFDAGEHARMEAAFAGPQGPMMNNASGFTPAEFARFQQ
QSRAGMPQTANHVASAPSPMMAGYQRPMGMGGYMGMGGMGMMPQTFN
PMAMQQQPAEATTQDKGKGRMVELDDENWEAQFAEMETADTQKLDDE
ANAAVEAELNDLDRSVPQDSGDSAFESVWQRVQAETATNRKLAEGET
DFNIDDNLHMGEMGEWDGFDTLNTRFRNPQLGDYMFEEDNVFRSVSN
PFEEGVKIMREGGNLSLAALAFEAAVQKDPQHVQAWTMLGSAQAQNE
KELPAIRALEQALKIDANNLDALMGLAVSYTNEGYDSTSYRTLERWL
SVKYPQIINPNDVSSEADLGFTDRQLLHDRVTDLFIQAAQLSPSGEQ
MDPDVQVGLGVLFYCAEEYDKAVDCFSAALASTESGTSNQQEQLHLL
WNRLGATLANSGRSEEAIEAYEQALNINPNFVRARYNLGVSCINIGC
YPEAAQHLLGALSMHRVVEQEGRERAREIVGGEGGIDDEQLDRMIHV
SQNQSTNLYDTLRRVFSQMGRRDLADQVVAGMDVNVFRREFEF

We claim:

1. A spore which is genetically modified or genetically engineered by a genetic DNA construct, wherein the genetic DNA construct comprises a first DNA portion encoding a target protein which is a bioactive polypeptide and a second DNA portion encoding a carrier, which construct, when transcribed and translated, expresses a fusion protein between the carrier and the target protein, characterized in that the spore is a strain of Bacillus subtilis and wherein the second DNA portion of the construct encoding the carrier is the spore structural gene cotD (encoding spore inner coat protein CotD) and wherein the spore is a viable spore which is able to germinate wherein said spore is genetically modified to produce an enzyme and/or a bioactive polypeptide upon germination into a vegetative cell.

2. The spore according to claim 1, wherein the bioactive polypeptide is a bacteriocin.

3. The spore according to claim 1, wherein the enzyme is phytase.
